(19) United States
(12) Patent Application Publication
(10) Pub. No.: US 2026/0265672 A1
(43) Pub. Date: Sep. 10, 2026

(54) SYSTEMS, METHODS, AND DEVICES FOR AUTOMATED CELLULAR THERAPY MANUFACTURING AND LIQUID CONTROL

(71) Applicant: Multiply Labs Inc., San Francisco, CA (US)

(72) Inventors: Michelle X. CHEN, San Francisco, CA (US); Chris CHENG, San Francisco, CA (US); Brigitte SCHMITTLEIN, San Francisco, CA (US); Dorothy SZYMKIEWICZ, San Francisco, CA (US); Dalind WILSON, San Francisco, CA (US); Jayna WITTENBRINK, San Francisco, CA (US)

(21) Appl. No.: 19/558,418

(22) Filed: Mar. 5, 2026

Related U.S. Application Data
(60) Provisional application No. 63/767,504, filed on Mar. 5, 2025.

(51) Int. Cl. C12M 1/36; C12M 1/00

(52) U.S. Cl. CPC C12M 41/48; C12M 29/04

(57) ABSTRACT

Systems, methods, and devices for control of a liquid are provided. An apparatus includes a housing having a first surface and a second surface. The first surface includes a first port disposed at a first height. The second surface includes a second port defining a first chamber. The second port is defined at a second height less than the first height. A first channel is disposed in an interior of the housing. The first channel has a first end portion in fluidic communication with the first port and a second end portion in fluidic communication with a first portion of the first chamber. A second channel is disposed in the interior of the housing. The second channel has a first end portion in fluidic communication with a second portion of the first chamber and a second end portion in fluidic communication with a first portion of a second chamber.

### CROSS-REFERENCE TO RELATED APPLICATION

The present Application claims priority to United States Provisional Patent Application No.: 63/767,504, entitled “Systems, Methods, and Devices for Automated Cellular Therapy Manufacturing and Liquid Control,” filed Mar. 5, 2025, which is hereby incorporated by reference in its entirety for all purposes.

### TECHNICAL FIELD

The present invention is directed to systems, methods, and devices for liquid manipulation and automated cellular therapy manufacture.

### BACKGROUND

Traditionally, cell therapies are produced with labor-intensive processes. These conventional processes require not only a large number of manufacturing operators, but also the employment of highly skilled (and expensive) technicians. These constraints make it particularly difficult to manufacture cell therapies at an industrial scale.

For instance, a large number of conventional solutions for manufacturing cell therapies have been focused on optimization of subsystems and/or more precise, real-time data collection, and a greater level of automation and integration of advanced robotics technologies. See, Rüβmann et al., 2015, “Industry 4.0: The future of productivity and growth in manufacturing industries,” Boston Consulting Group, 9(1), 54.

Furthermore, fluids and other reagents utilized during the manufacturing of cell therapies have strict sterilization and contamination protocols and requirements that must be followed to ensure satisfactory quality of the manufactured cell therapy, which is difficult to ensure using human or automated processes. For instance, some such fluids include live cells and/or reagents that are sensitive to light and/or moisture, further complicating the manufacturing of cell therapies utilizing such fluids.

Given the above background, what is needed in the art are improved systems, methods, and devices for automated manufacture of cell therapies.

### SUMMARY

The present disclosure addresses the shortcomings disclosed above and other problems associated with user interfaces for electronic devices (or more generally, computer systems) by providing systems, methods, and devices that facilitate automated manufacture of one or more cellular therapies.

In some embodiments, the systems, methods, and devices of the present disclosure allows for the automated manufacture of one or more cell therapies by controlling a fluid.

Turning to more specific aspects, the present disclosure provides an apparatus for controlling a liquid. In some embodiments, the apparatus includes a housing. The housing includes a first surface and a second surface different from the first surface. The first surface includes a first port disposed at a first height. The second surface includes a second port defining a first chamber. The second port is defined at a second height, which is less than the first height. A first channel is disposed in an interior of the housing. The first channel includes a first end portion in fluidic communication with the first port and a second end portion in fluidic communication with a first portion of the first chamber. A second channel is disposed in the interior of the housing. The second channel includes a first end portion in fluidic communication with a second portion of the first chamber and a second end portion in fluidic communication with a first portion of a second chamber.

In some embodiments, the first port includes a first diameter configured to receive an end portion of a first device. The first device is configured to control a flow of the liquid through the housing.

In some embodiments, the second port includes a second diameter configured to receive an end portion of a second device. The second device is configured to receive a flow of the liquid through the housing.

In some embodiments, the second port is configured to accommodate a gasket that includes a first surface configured to interface with an exterior surface of the second port and a second surface configured to interface with an exterior surface of a second device.

In some embodiments, the first chamber defines a first reservoir that includes an interior volume configured to accommodate both the liquid and a second device.

In some embodiments, the second chamber defines a second reservoir that includes a closed-form shape.

In some embodiments, the first end portion of the second channel includes a first height and the second end portion of the second channel includes a second height less than the first height.

In some embodiments, the first channel defines a continuous fluid flow path between an upstream portion at the first port and a downstream portion at the first portion of the first chamber.

In some embodiments, the first channel extends monotonically in a first direction relative to a gravitational vector.

In some embodiments, the first channel extends downwardly relative to a gravitational vector when the apparatus is (i) horizontal with the ground and/or (ii) rotated about a first axis of the housing by at least 45 degrees.

In some embodiments, the second channel defines a continuous fluid flow path between an upstream portion at the second portion of the first chamber and a downstream portion at the first portion of the second chamber.

In some embodiments, the second channel extends monotonically in a first direction relative to a gravitational vector.

In some embodiments, a first interior volume of the first chamber is greater than a second interior volume of the second chamber.

In some embodiments, the first portion of the second chamber includes the sole fluid opening of the second chamber.

In some embodiments, the apparatus further includes a third channel disposed in the interior of the housing. The third channel includes a first end portion in fluidic communication with the second portion of the first chamber and a second end portion in fluidic communication with a third opening of the housing.

In some embodiments, the third opening is formed in an interior of the housing.

In some embodiments, the third opening is configured to accommodate a filter mechanism.

In some embodiments, the first channel includes a tubing. The second channel is formed by at least a first interior surface of the housing.

In some embodiments, the housing is configured to mate to a corresponding external surface of a third device.

### BRIEF DESCRIPTION OF THE DRAWINGS

FIG. 1 illustrates various modules and/or components of a computer system, in accordance with an embodiment of the present disclosure.

FIG. 2 illustrates a schematic view of a biological foundry for manufacturing one or more cellular therapies using one or more instruments including an articulated handling robot for interfacing with an apparatus to control a liquid associated with the apparatus, in accordance with an embodiment of the present disclosure.

FIG. 3 illustrates various logic functions that are implemented by a computer system in some embodiments of the present disclosure.

FIG. 4 is a perspective view illustrating an exemplary apparatus in accordance with some exemplary embodiments of the present disclosure.

FIG. 5 is a cross-sectional view of the exemplary apparatus of FIG. 4.

FIG. 6 is a perspective view illustrating another exemplary apparatus in accordance with some exemplary embodiments of the present disclosure.

FIG. 7 is a cross-sectional view of the exemplary apparatus of FIG. 6.

FIG. 7 is another cross-sectional view of the exemplary apparatus of FIG. 6.

FIG. 9 is a perspective view illustrating an interior of an exemplary apparatus in accordance with some exemplary embodiments of the present disclosure.

FIG. 10 is a perspective view illustrating another exemplary apparatus in accordance with some exemplary embodiments of the present disclosure.

FIG. 11 is a perspective view illustrating an interior of another exemplary apparatus in accordance with some exemplary embodiments of the present disclosure.

FIG. 12 is an enlarged view of the exemplary apparatus of FIG. 11.

FIG. 13 is a perspective view illustrating another exemplary apparatus in accordance with some exemplary embodiments of the present disclosure.

FIGS. 14A-14E collectively illustrate a process of controlling flow of a liquid using an exemplary apparatus in accordance with some exemplary embodiments of the present disclosure.

FIGS. 15-21 collectively illustrate another process of controlling flow of a liquid using an exemplary apparatus in accordance with some exemplary embodiments of the present disclosure.

FIG. 22 is a perspective view illustrating a surface of an exemplary apparatus in accordance with some exemplary embodiments of the present disclosure.

FIG. 23 is another perspective view illustrating a surface of an exemplary apparatus in accordance with some exemplary embodiments of the present disclosure.

FIG. 24 is a side view illustrating an exemplary apparatus in accordance with some exemplary embodiments of the present disclosure.

FIG. 25 is another side view illustrating an exemplary apparatus in accordance with some exemplary embodiments of the present disclosure.

FIG. 26 is a view of an exemplary apparatus in accordance with some exemplary embodiments of the present disclosure.

FIG. 27 is a fragmentary view of an exemplary apparatus of FIG. 26 in accordance with some exemplary embodiments of the present disclosure.

FIG. 28 is a rear view of an exemplary apparatus in accordance with some exemplary embodiments of the present disclosure.

FIGS. 29A-29C collectively illustrate an exemplary second chamber and filter mechanism of an exemplary apparatus in accordance with some exemplary embodiments of the present disclosure.

FIGS. 30A-30C collectively illustrate another exemplary second chamber and filter mechanism of an exemplary apparatus in accordance with some exemplary embodiments of the present disclosure.

FIGS. 31A-31C collectively illustrate yet another exemplary second chamber and a filter mechanism of an exemplary apparatus in accordance with some exemplary embodiments of the present disclosure.

FIGS. 32A and 32B are perspective views illustrating another exemplary apparatus in accordance with some exemplary embodiments of the present disclosure.

FIG. 33 is a cross-sectional view of the exemplary apparatus.

FIG. 34 is a perspective view illustrating the side profile of the apparatus.

FIG. 35 is a perspective view illustrating another exemplary apparatus in accordance with some exemplary embodiments of the present disclosure.

FIG. 36 is another perspective view illustrating another exemplary apparatus in accordance with some exemplary embodiments of the present disclosure.

FIG. 37 is another perspective view illustrating another exemplary apparatus in accordance with some exemplary embodiments of the present disclosure.

FIG. 38 is a perspective view illustrating an exemplary apparatus in accordance with some exemplary embodiments of the present disclosure.

FIG. 39 is a perspective view illustrating a profile of another exemplary apparatus in accordance with some exemplary embodiments of the present disclosure.

FIG. 40 is a cross-sectional view of an exemplary apparatus in accordance with some exemplary embodiments of the present disclosure.

FIG. 41 is a perspective view illustrating an exemplary apparatus in accordance with some exemplary embodiments of the present disclosure.

FIG. 42 is a perspective view illustrating an exemplary apparatus in accordance with some exemplary embodiments of the present disclosure.

FIG. 43 is a cross-sectional view of the exemplary apparatus of FIG. 42.

FIGS. 44A-44B collectively illustrate an enlarged view of an exemplary apparatus in accordance with some exemplary embodiments of the present disclosure.

FIG. 45 is a cross-sectional view illustrating an exemplary apparatus in accordance with some exemplary embodiments of the present disclosure.

FIG. 46 is another cross-sectional view illustrating an exemplary apparatus in accordance with some exemplary embodiments of the present disclosure.

FIG. 47 is a perspective view illustrating an interior of an exemplary apparatus in accordance with some exemplary embodiments of the present disclosure.

FIG. 48 is a perspective view illustrating an exemplary apparatus in accordance with some exemplary embodiments of the present disclosure.

FIG. 49 is an enlarged view illustrating an exemplary apparatus in accordance with some exemplary embodiments of the present disclosure.

FIG. 50 is another enlarged view illustrating an exemplary apparatus in accordance with some exemplary embodiments of the present disclosure.

FIG. 51 is an enlarged perspective view illustrating an exemplary apparatus in accordance with some exemplary embodiments of the present disclosure.

FIG. 52 is another enlarged perspective view illustrating an exemplary apparatus in accordance with some exemplary embodiments of the present disclosure.

FIG. 53 is an enlarged perspective view illustrating an exemplary apparatus in accordance with some exemplary embodiments of the present disclosure.

FIG. 54 is another enlarged perspective view illustrating an exemplary apparatus in accordance with some exemplary embodiments of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Systems, methods, and devices for facilitating automated manufacture of one or more cellular therapies through control of a liquid are provided. For instance, in some embodiments, the systems, methods, and devices of the present disclosure allow for controlling a liquid at a biological foundry during a workflow to manufacture some or all of a cellular therapy.

In some embodiments, the present disclosure provides an apparatus for controlling a flow of a liquid. In some embodiments, the apparatus includes a housing having a first surface and a second surface. The first surface includes a first port disposed at a first height. The second surface includes a second port defining a first chamber. The second port is defined at a second height less than the first height. A first channel is disposed in an interior of the housing. The first channel has a first end portion in fluidic communication with the first port and a second end portion in fluidic communication with a first portion of the first chamber. A second channel is disposed in the interior of the housing. The second channel has a first end portion in fluidic communication with a second portion of the first chamber and a second end portion in fluidic communication with a first portion of a second chamber. Accordingly, the apparatus of the present disclosure provides a fluid pathway extending between two different devices (e.g., a first device suppling a liquid and a second device receiving a liquid, a first device supplying and receiving a liquid, a second device receiving the liquid, etc.).

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first property could be termed a second property, and, similarly, a second property could be termed a first property, without departing from the scope of the present disclosure. The first property and the second property are both properties, but they are not the same property.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case-and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, +2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed. Alternatively, particularly with respect to biological systems or processes, the term can mean within an order of magnitude, e.g., within 5-fold, or within 2-fold, of a value.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal Instruction Set Computers (MISC), Very Long Instruction Word (VLIW), Explicitly Parallel Instruction Computing (EPIC), and One Instruction Set Computer (OISC).

Furthermore, when a reference number is given an “i^{th}” denotation, the reference number refers to a generic component, set, or embodiment. For instance, a device termed “device i” refers to the i^{th} device in a plurality of devices (e.g., a device 100-i in a plurality of devices 100).

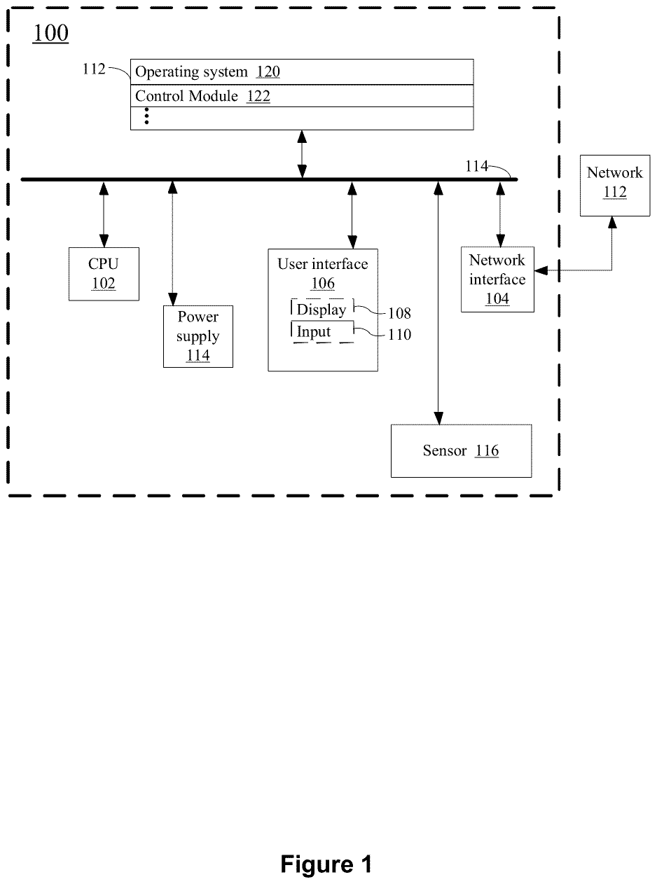

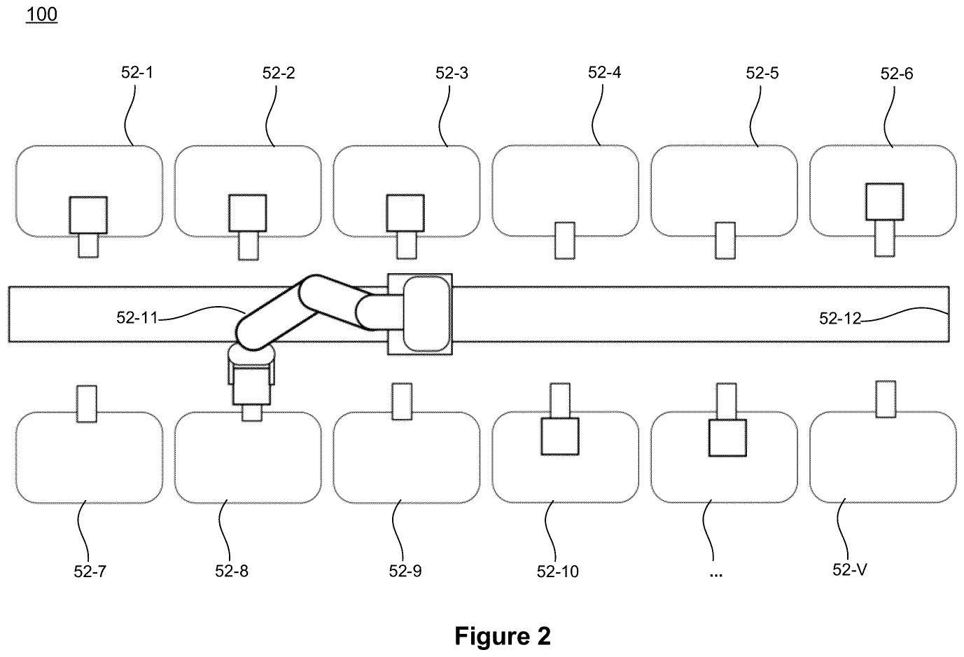

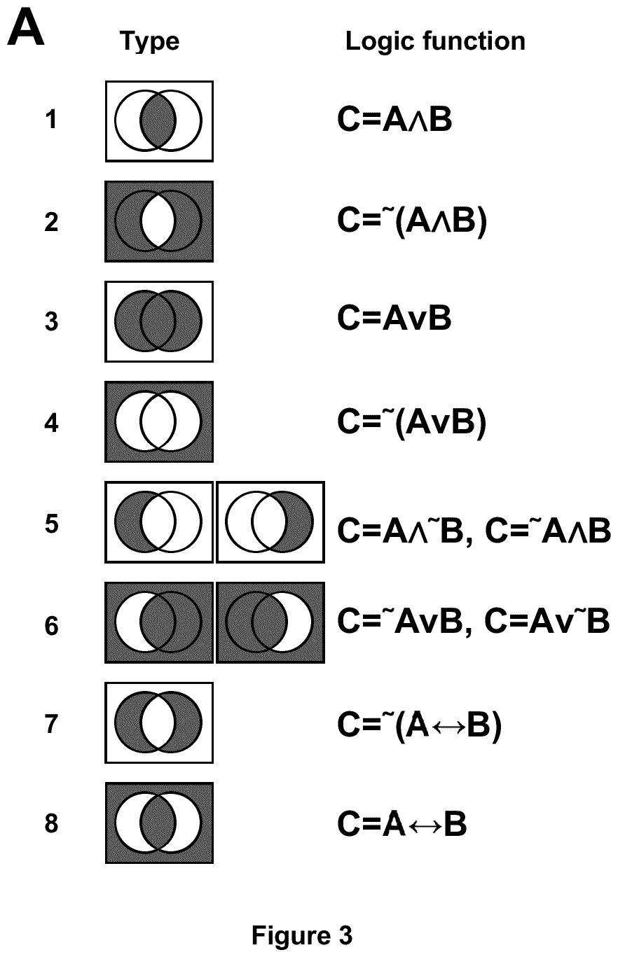

In the present disclosure, unless expressly stated otherwise, descriptions of devices and systems will include implementations of one or more computers. For instance, and for purposes of illustration in FIG. 1, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 112 of FIG. 1). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 112, the illustrated devices and systems may wirelessly transmit information between each other. As such, the exemplary topology shown in FIG. 1 merely serves to describe the features of an embodiment of the present disclosure in a manner that will be readily understood to one of skill in the art.

FIG. 1 depicts a block diagram of a distributed computer system (e.g., computer system 100) according to some embodiments of the present disclosure. The computer system 100 at least facilitates communicating one or more instructions for facilitating automated manufacture of one or more cellular therapies and/or control of a portion of a device.

In some embodiments, the communication network 112 optionally includes the Internet, one or more local area networks (LANs), one or more wide area networks (WANs), other types of networks, or a combination of such networks.

Examples of communication networks 112 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 102, a network or other communications interface 104, and memory 112.

In some embodiments, the computer system 100 includes a user interface 106. The user interface 106 typically includes a display 108 for presenting media. In some embodiments, the display 108 is integrated within the computer systems (e.g., housed in the same chassis as the CPU 102 and memory 112). In some embodiments, the computer system 100 includes one or more input device(s) 110, which allow a subject to interact with the computer system 100. In some embodiments, input devices 110 include a keyboard, a mouse, and/or other input mechanisms. Alternatively, or in addition, in some embodiments, the display 108 includes a touch-sensitive surface (e.g., where display 108 is a touch-sensitive display or computer system 100 includes a touch pad).

In some embodiments, the computer system 100 presents media to a user through the display 108. Examples of media presented by the display 108 include one or more images, a video, audio (e.g., waveforms of an audio sample), or a combination thereof. In typical embodiments, the one or more images, the video, the audio, or the combination thereof is presented by the display 108 through a client application (e.g., client application 124 of FIG. 1). In some embodiments, the audio is presented through an external device (e.g., speakers, headphones, input/output (I/O) subsystem, etc.) that receives audio information from the computer system 100 and presents audio data based on this audio information. In some embodiments, the user interface 106 also includes an audio output device, such as speakers or an audio output for connecting with speakers, earphones, or headphones.

Memory 112 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 112 may optionally include one or more storage devices remotely located from the CPU(s) 102. Memory 112, or alternatively the non-volatile memory device(s) within memory 112, includes a non-transitory computer readable storage medium. Access to memory 192 by other components of the computer system 100, such as the CPU(s) 102, is, optionally, controlled by a controller. In some embodiments, memory 112 can include mass storage that is remotely located with respect to the CPU(s) 102. In other words, some data stored in memory 112 may in fact be hosted on devices that are external to the computer system 100, but that can be electronically accessed by the computer system 100 over an Internet, intranet, or other form of network 112 or electronic cable using communication interface 104.

In some embodiments, the memory 112 of the computer system 100 stores:
an operating system 120 (e.g., ANDROID, IOS, DARWIN, RTXC, LINUX, UNIX, OS X, WINDOWS, or an embedded operating system such as VxWorks) that includes procedures for handling various basic system services;
an optional electronic address associated with the computer system 100 that identifies the computer system 100 (e.g., within the communication network 112);
a control module 122 for controlling one or more instruments 52 (e.g., a metering device, a gripper device, a nest device, a calibration device, a docking device, a mounting device, a tray device, a tablet deposition device, or a combination thereof) associated with the computer system 100; and
a client application for presenting information (e.g., media) using a display 108 of the computer system 100.

As indicated above, in some embodiments, an electronic address is associated with the computer system 100. The electronic address is utilized to at least uniquely identify the computer system 100 from other devices and components of the distributed system 100, such as other devices having access to the communications network 112.

In some embodiments, a control module 122 is utilized for controlling one or more controlling one or more instruments 52 (e.g., a metering device, a gripper device, a nest device, a calibration device, a docking device, a mounting device, a tray device, a tablet deposition device, or a combination thereof) associated with the computer system 100 and/or for controlling performance of one or more workflows at the one or more instruments 52. For instance, in some embodiments, the one or more instruments 52 includes an articulated handling robot 52-11 that is configured to advance the one or more workflows between a first instrument and a second instrument in the one or more instruments at a biological foundry. However, one of skill in the art will appreciate that the present disclosure is not so limited.

In some embodiments, the computer system optionally also includes one or more sensors 116, such as one or more optional sensors 116 to allow for visual inspection of a respective instrument. The optical sensor(s) optionally include charge-coupled device (CCD) or complementary metal-oxide semiconductor (CMOS) phototransistors. The optical sensor(s) optionally capture still images and/or video, such as an image of a first cellular therapy and/or a volume of a first container associated with the computer system.

In some embodiments, the systems, methods, and apparatuses of the present disclosure include one or more systems, methods, apparatuses, and devices of United States Patent Publication no.: 2023-0102750 A1, entitled “SYSTEMS AND METHODS FOR FACILITATING MODULAR AND PARALLELIZED MANUFACTURING AT A BIOLOGICAL FOUNDRY,” filed Aug. 17, 2022; United States Patent Publication no.: 2022-0325219 A1, entitled “SYSTEM, METHOD, AND APPARATUS FACILITATING AUTOMATED MODULAR MANUFACTURE OF CELL THERAPY,” filed Dec. 13, 2021; United States Patent Publication no.: 2019-0016048 A 1, entitled “SYSTEMS AND METHODS FOR DESIGNING AND MANUFACTURING MULTI-COMPARTMENT CAPSULES,” filed Jun. 18, 2018; U.S. Pat. No. 11,198,845, entitled “SYSTEM, METHOD, AND APPARATUS FACILITATING AUTOMATED MODULAR MANUFACTURE OF CELL THERAPY,” filed Apr. 19, 2021; U.S. Pat. No.: 10,773,392, entitled “FLEXURE GRIPPING DEVICE,” filed Mar. 7, 2019; U.S. Pat. No.: 11,142,353; United States Patent no.: 9,845,167, entitled “AUTOMATED BATCH FILLING APPARATUS,” filed Mar. 7, 2019; U.S. Pat. No.: 10,456,975, entitled “MULTI-COMPARTMENT CAPSULE,” filed Jun. 16, 2017; U.S. patent application Ser. No. 18/306,936, entitled “SYSTEM, METHOD, AND APPARATUS FOR MANUFACTURE OF ENGINEERED CELLS,” filed Apr. 25, 2023; U.S. patent application Ser. No. 18/154,950, entitled “SYSTEM, METHOD, AND APPARATUS FOR MANUFACTURE OF ENGINEERED CELLS,” filed Jan. 16, 2023; U.S. patent application Ser. No. 17/823,065, entitled “SYSTEM, METHOD, AND APPARATUS FACILITATING ASSEMBLY OF A CAPSULE,” filed Aug. 29, 2022; and United States Patent Publication no.: 2024/0359848A 1 , entitled “SYSTEM, METHOD, AND APPARATUS FOR CAPSULE FABRICATION,” filed Apr. 29, 2024, each of which is hereby incorporated by reference in its entirety for all purposes.

Each of the above identified modules and applications correspond to a set of executable instructions for performing one or more functions described above and the methods described in the present disclosure (e.g., the computer-implemented methods and other information processing methods described herein). These modules (e.g., sets of instructions) need not be implemented as separate software programs, procedures or modules, and thus various subsets of these modules are, optionally, combined or otherwise re-arranged in various embodiments of the present disclosure. In some embodiments, the memory optionally stores a subset of the modules and data structures identified above. Furthermore, in some embodiments, the memory stores additional modules and data structures not described above.

It should be appreciated that the computer system of FIG. 1 is only one example, and that the computer system optionally has more or fewer components than shown, optionally combines two or more components, or optionally has a different configuration or arrangement of the components shown in FIG. 1 are implemented in hardware, software, firmware, or a combination thereof, including one or more signal processing and/or application specific integrated circuits.

Now that details of a computer system 100 for facilitating automated manufacture of one or more cellular therapies by controlling a liquid have been disclosed, details regarding one or more devices and/or one or more apparatuses for controlling a liquid is provided.

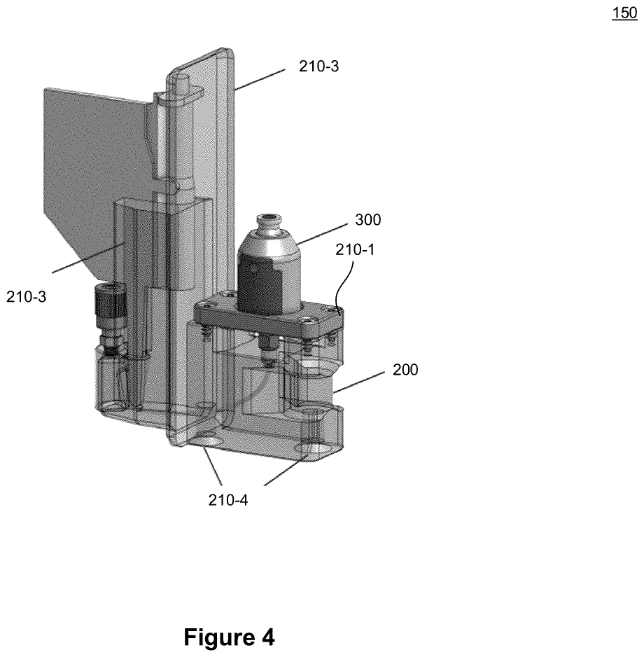

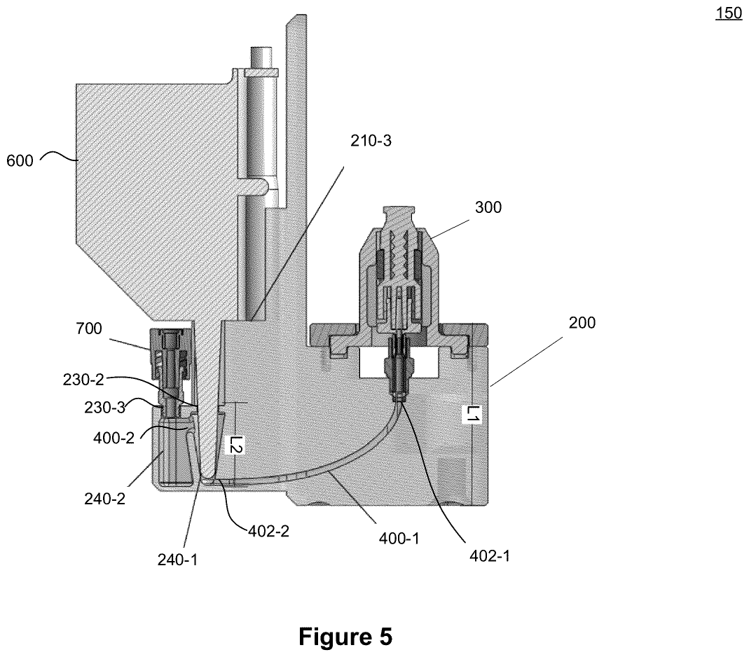

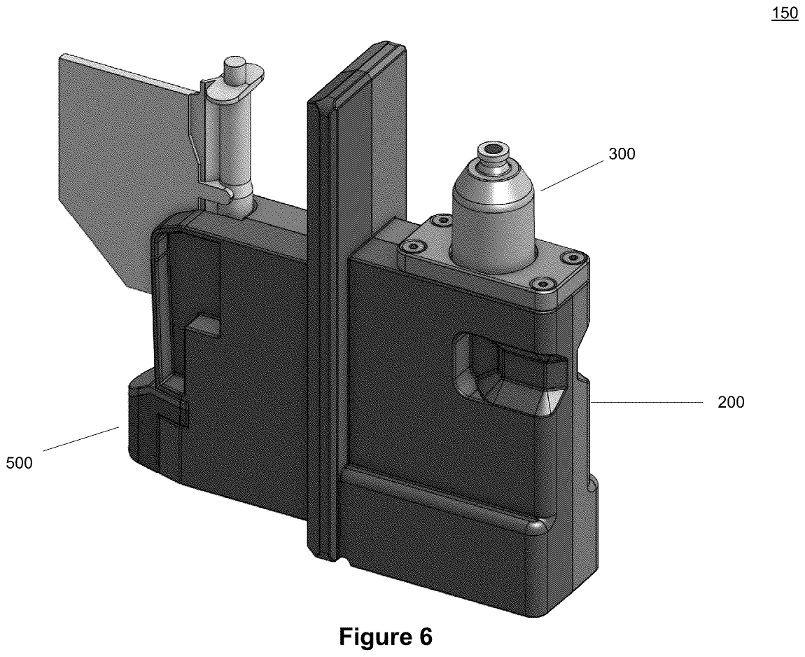

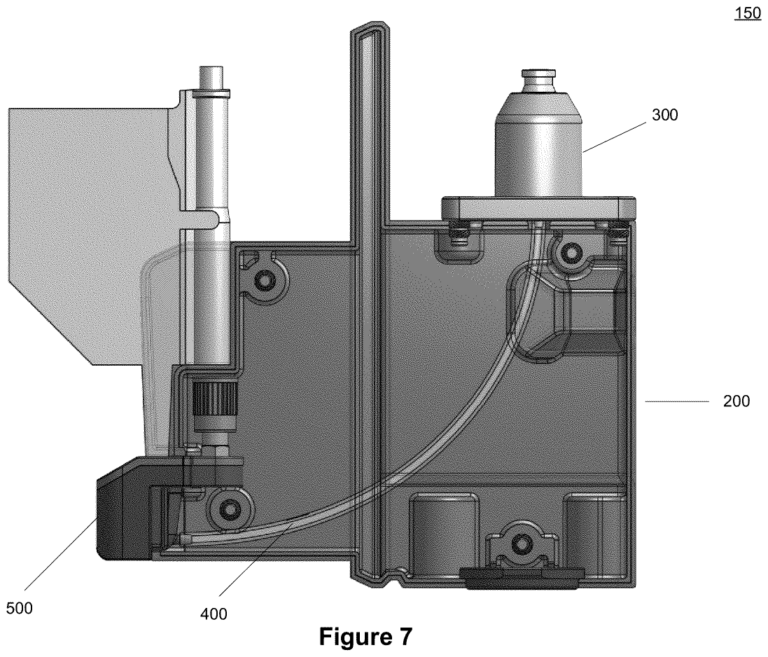

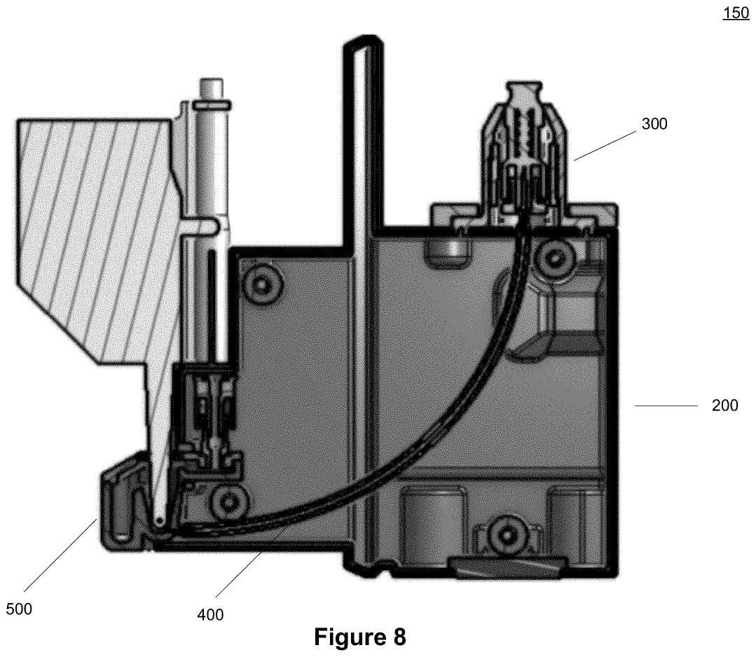

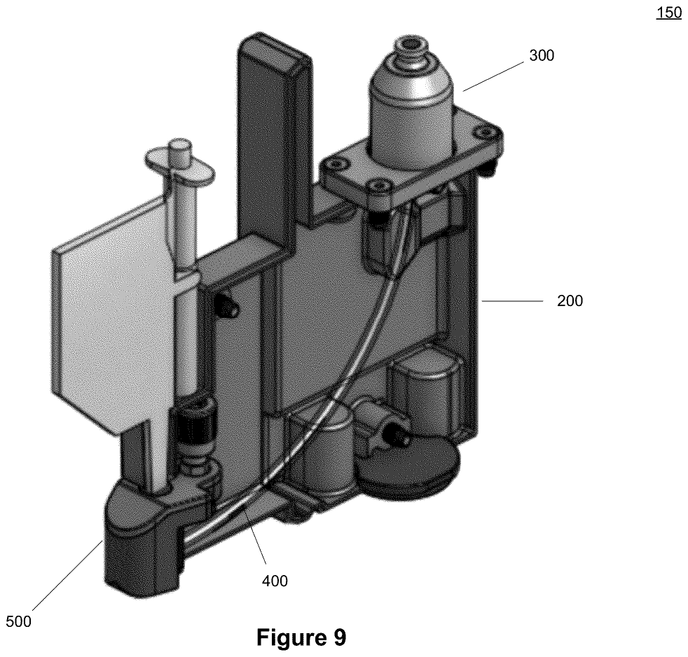

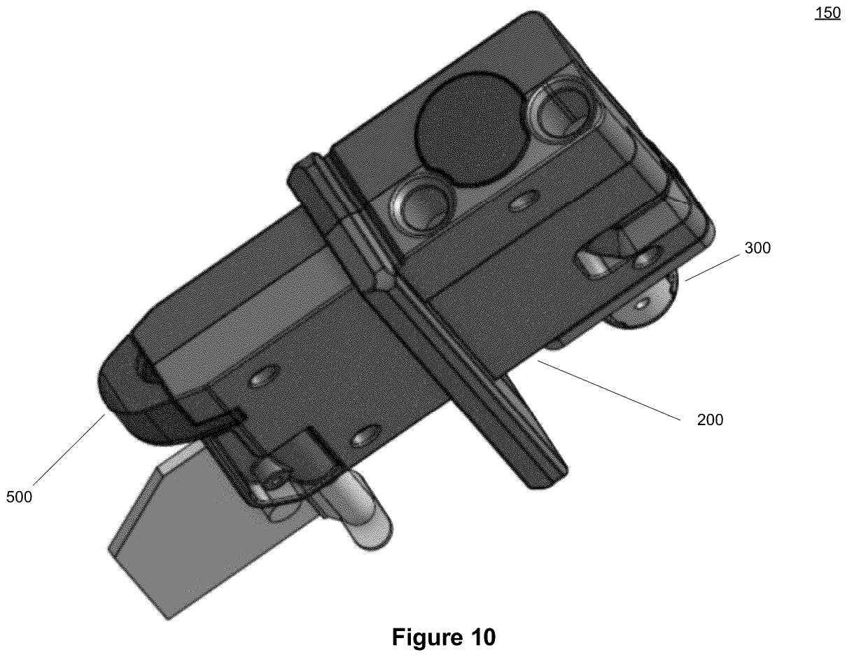

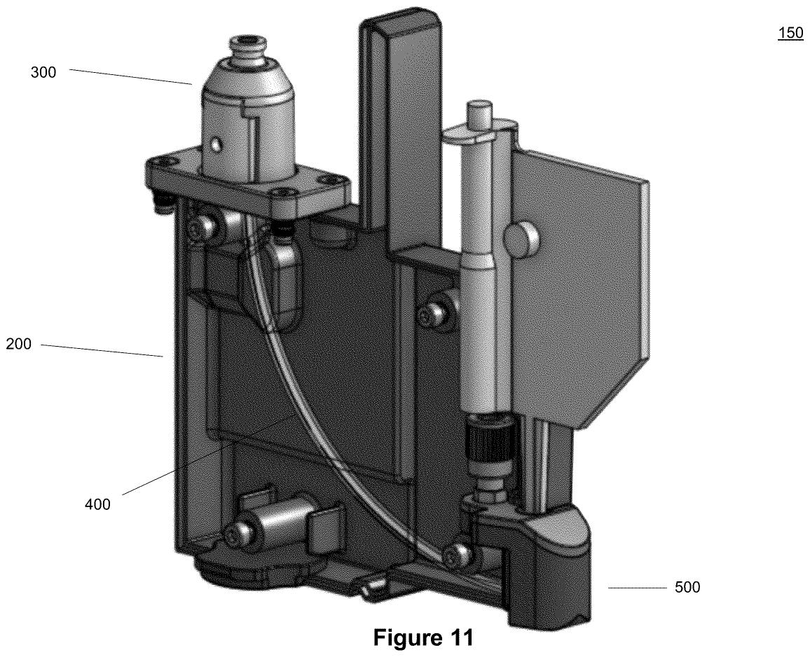

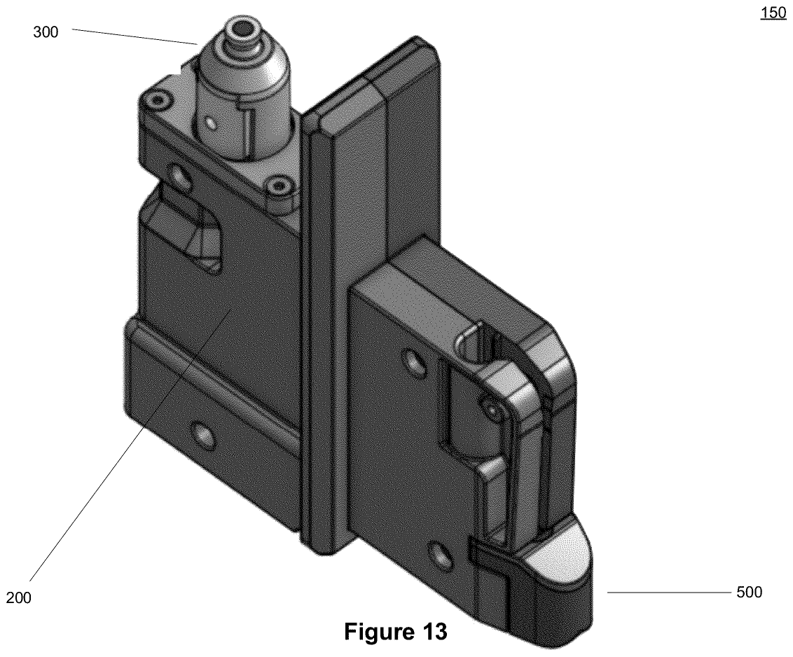

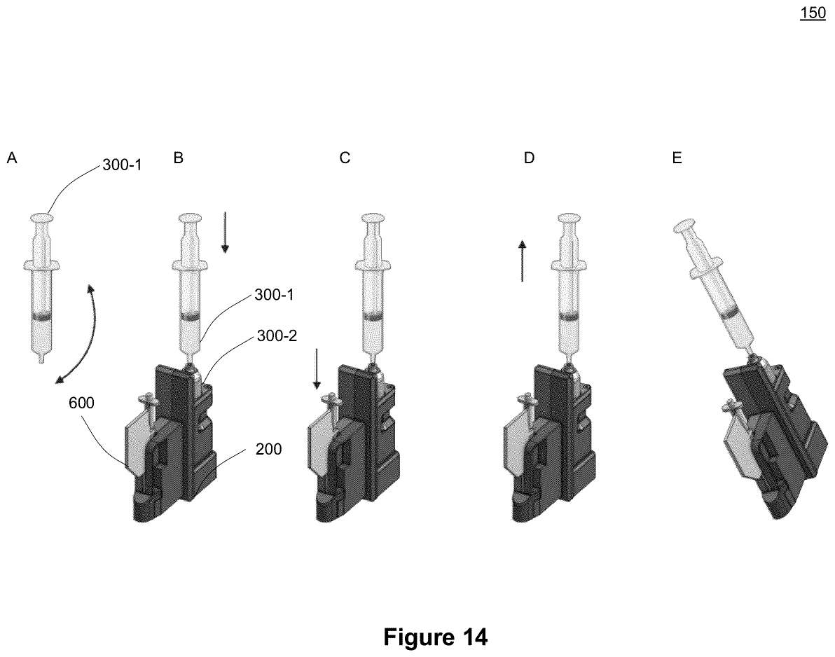

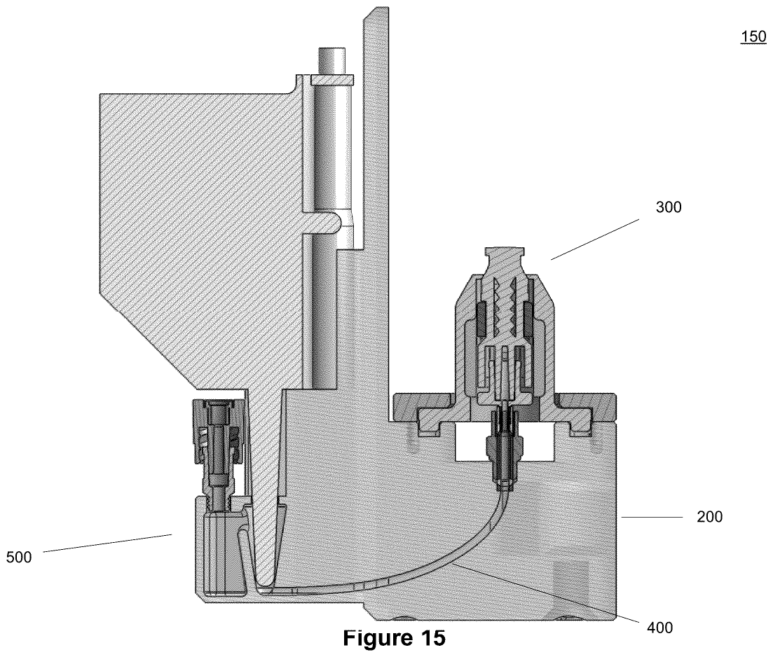

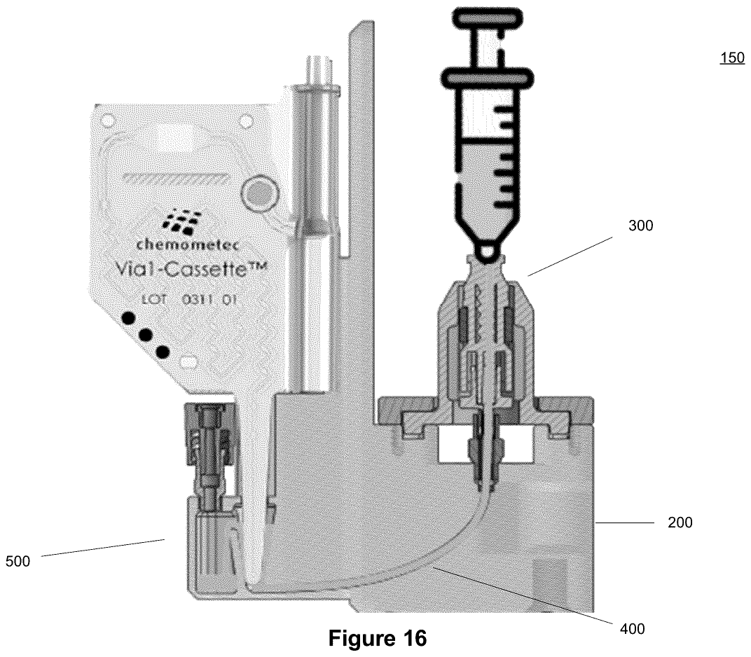

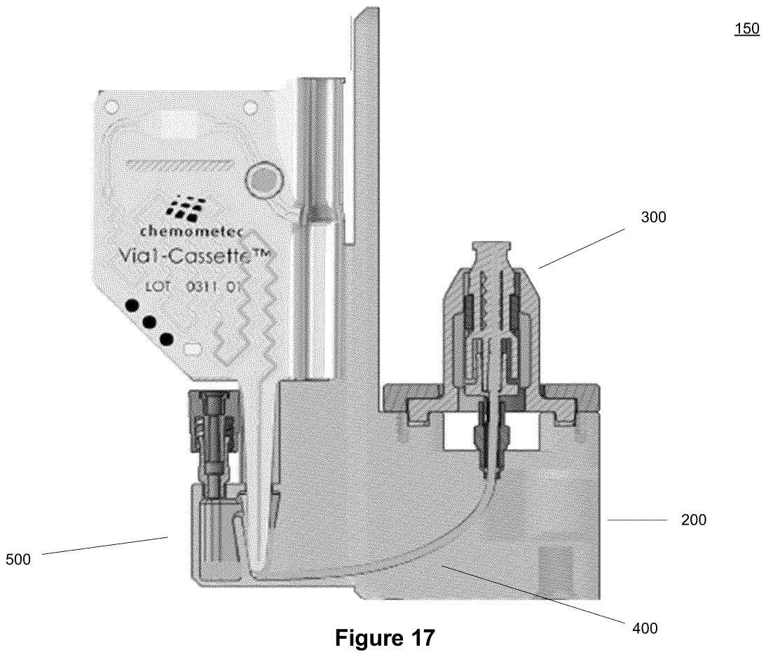

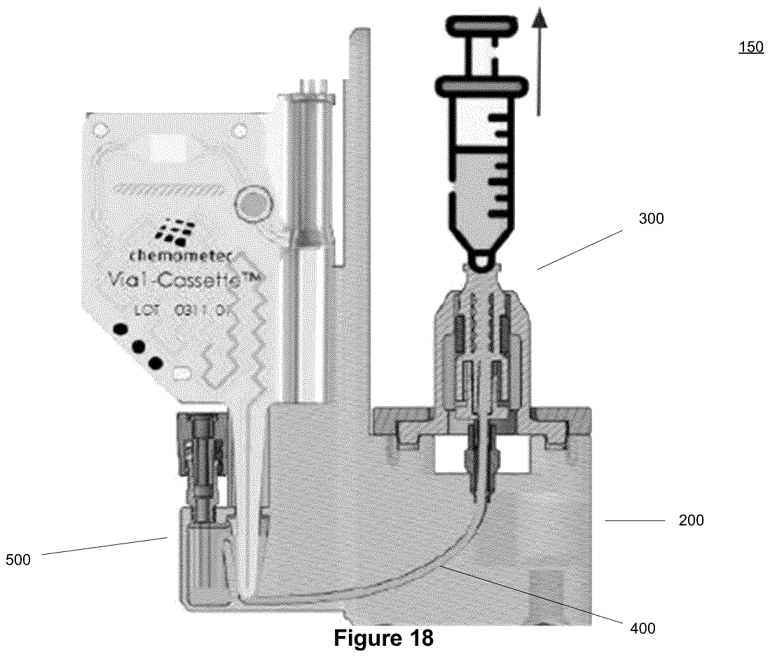

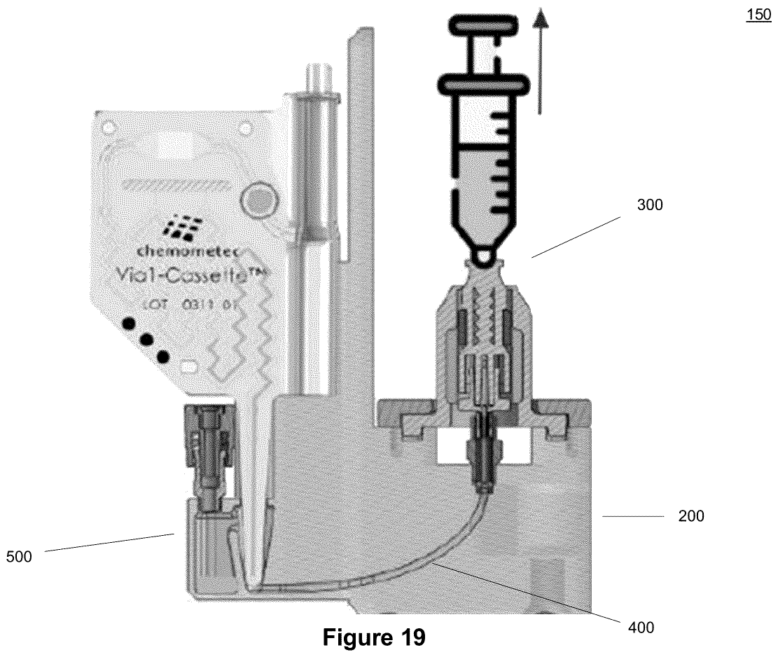

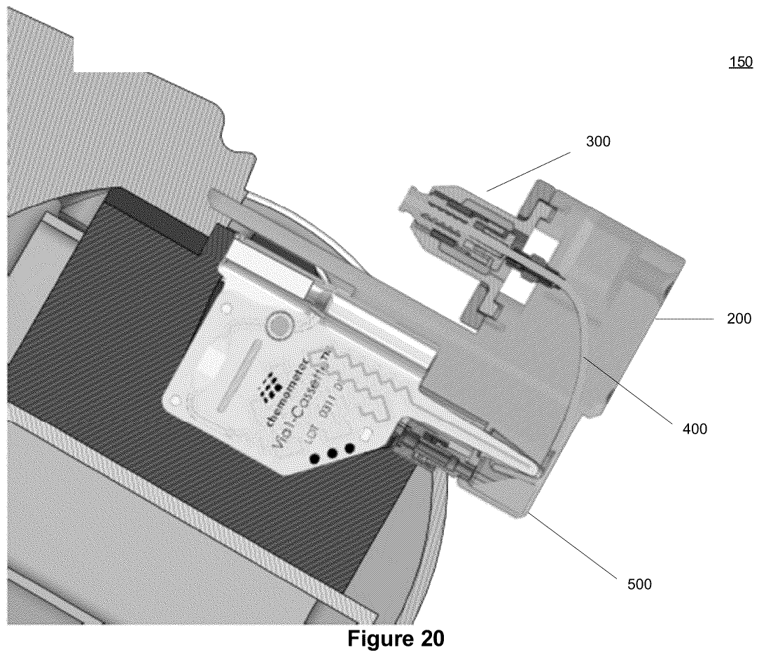

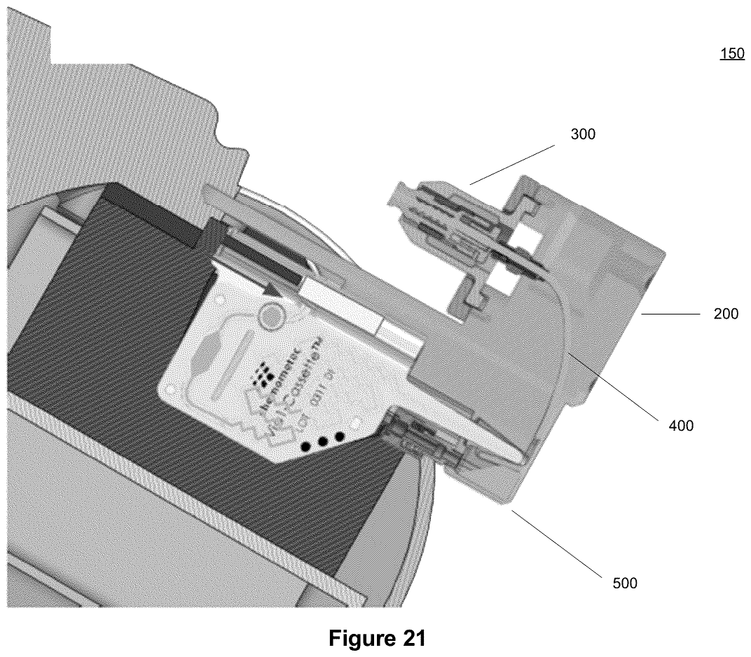

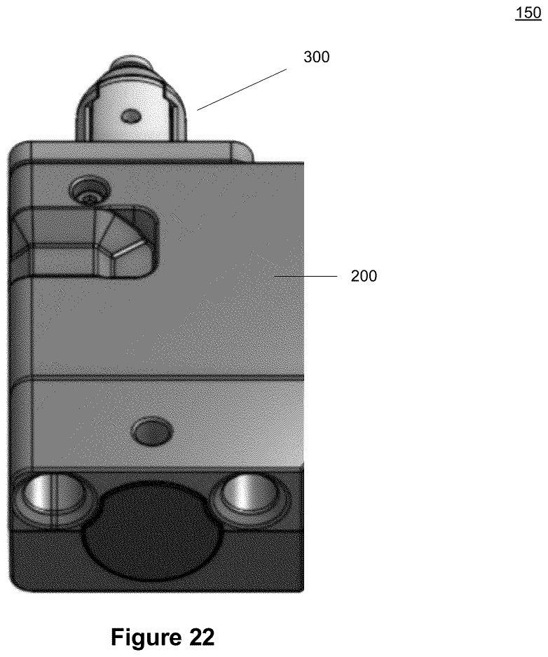

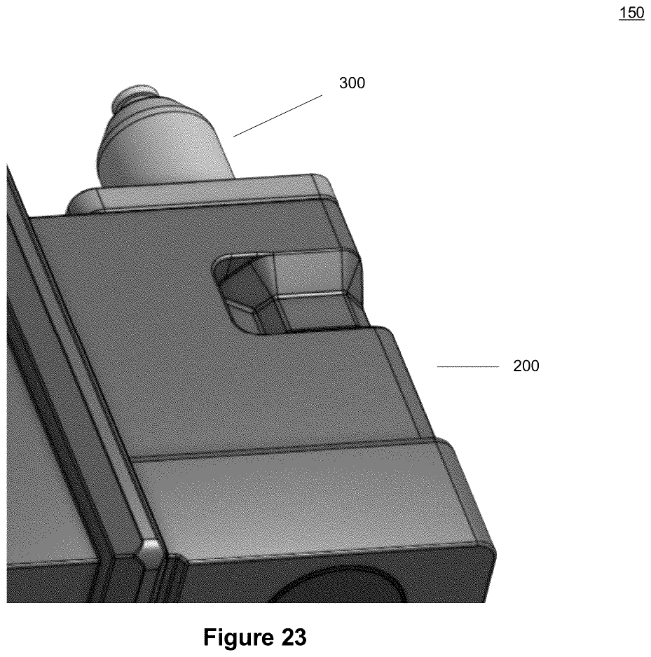

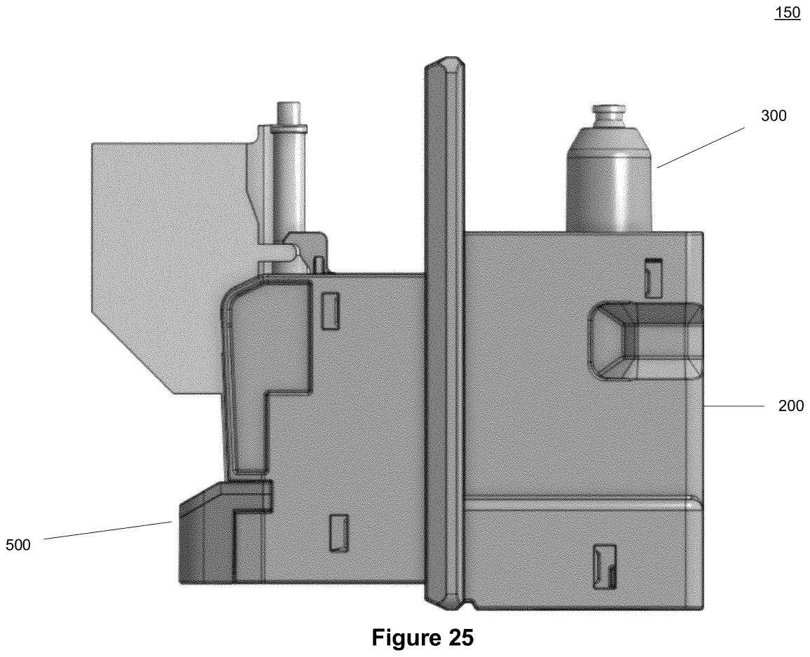

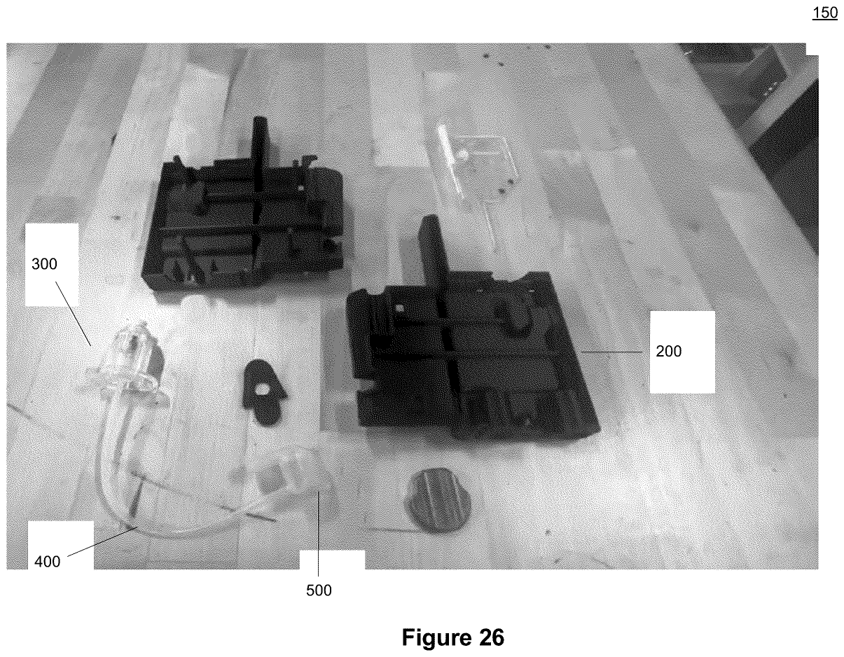

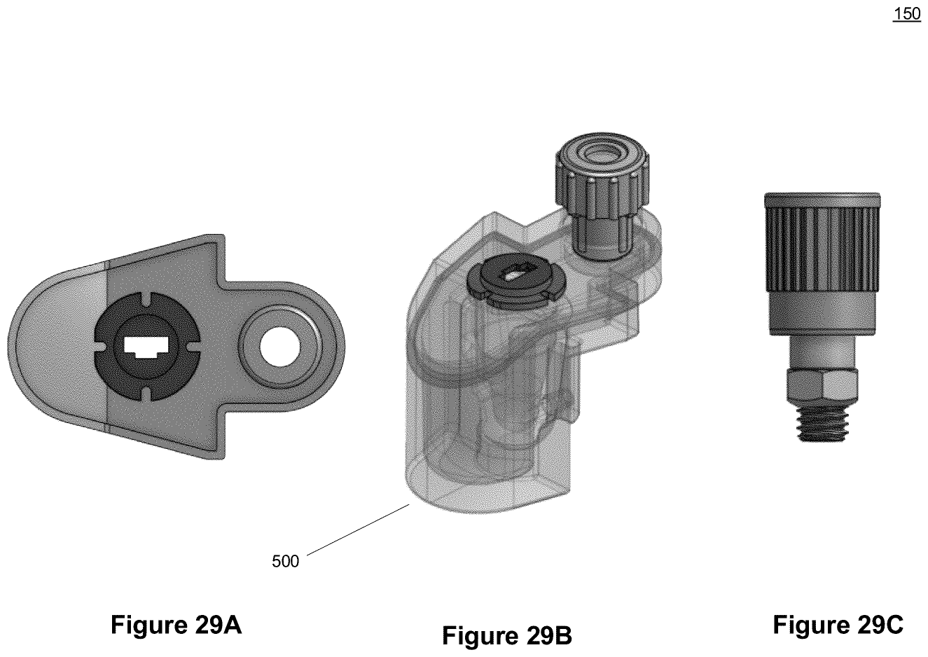

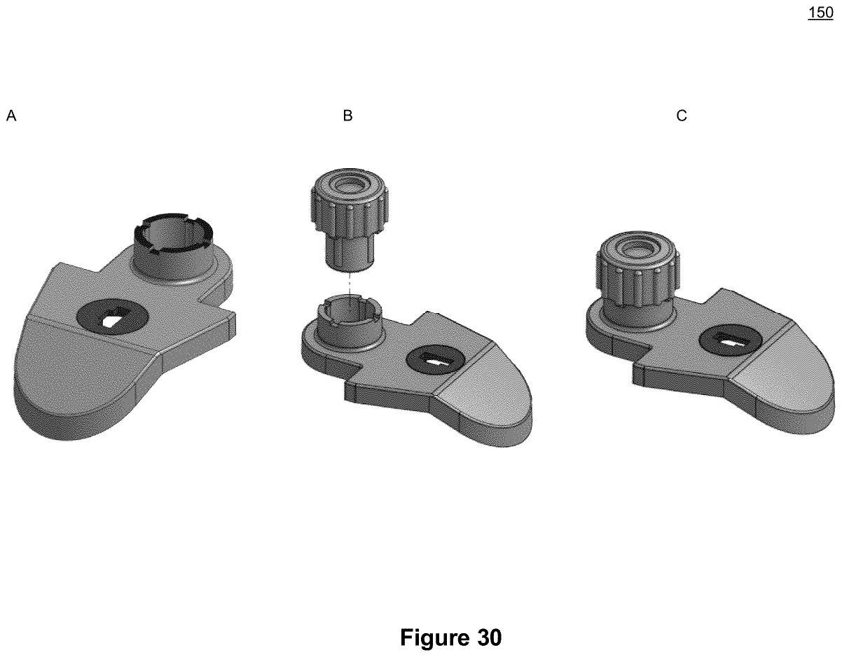

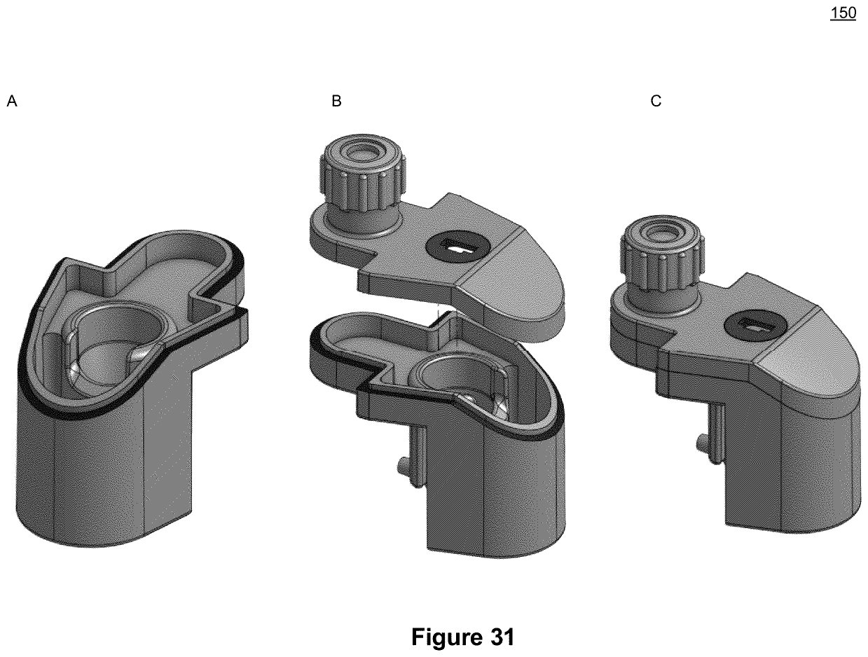

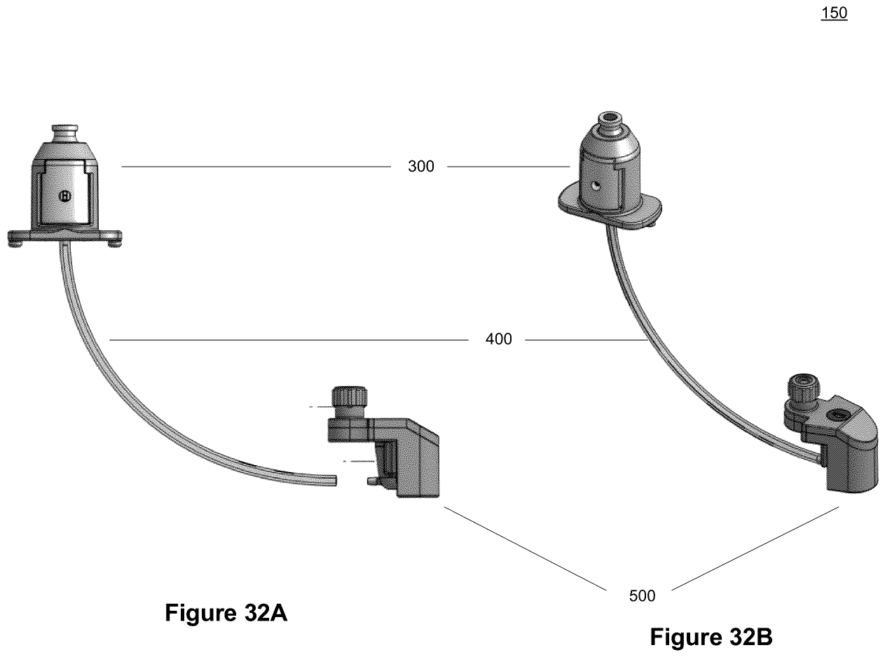

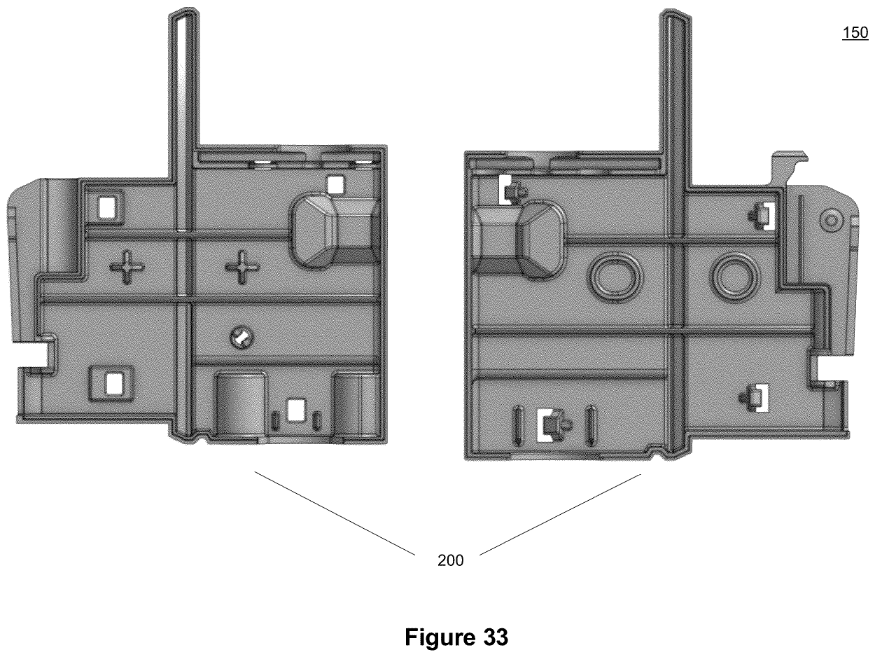

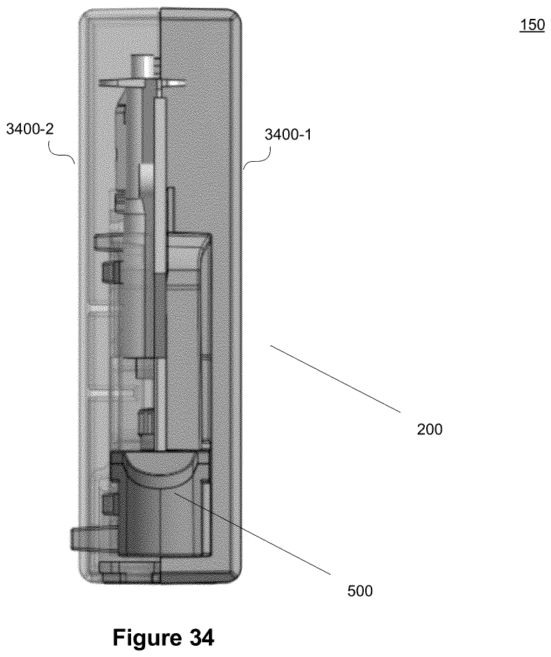

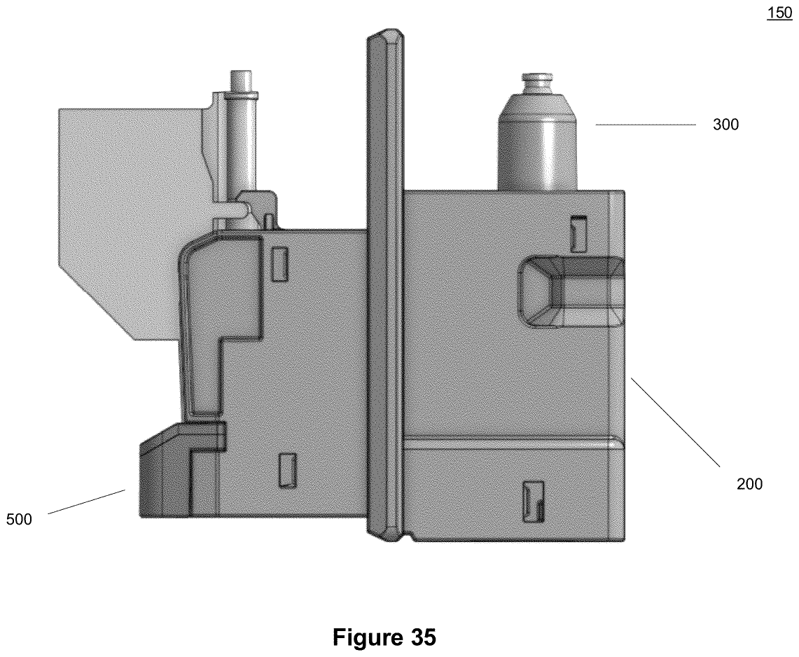

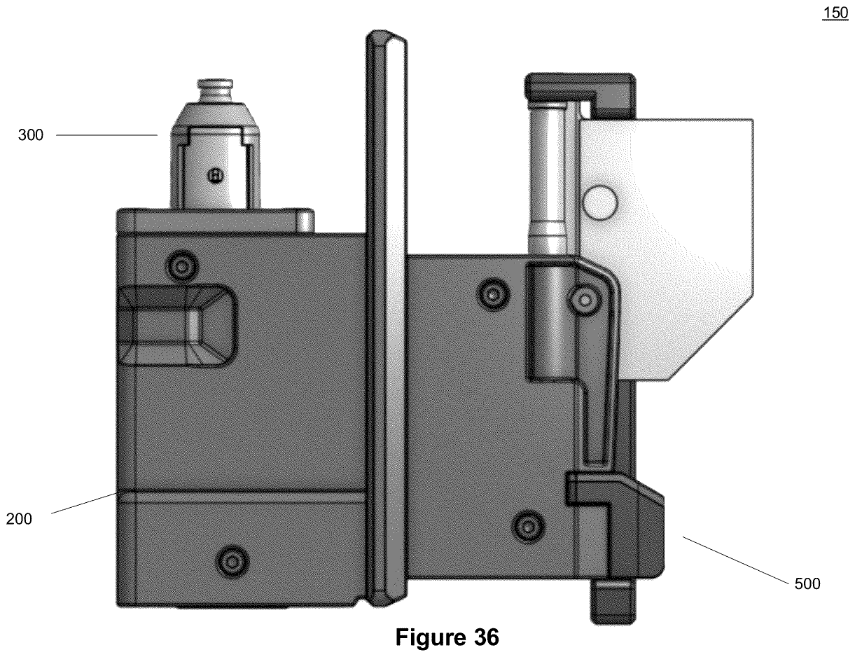

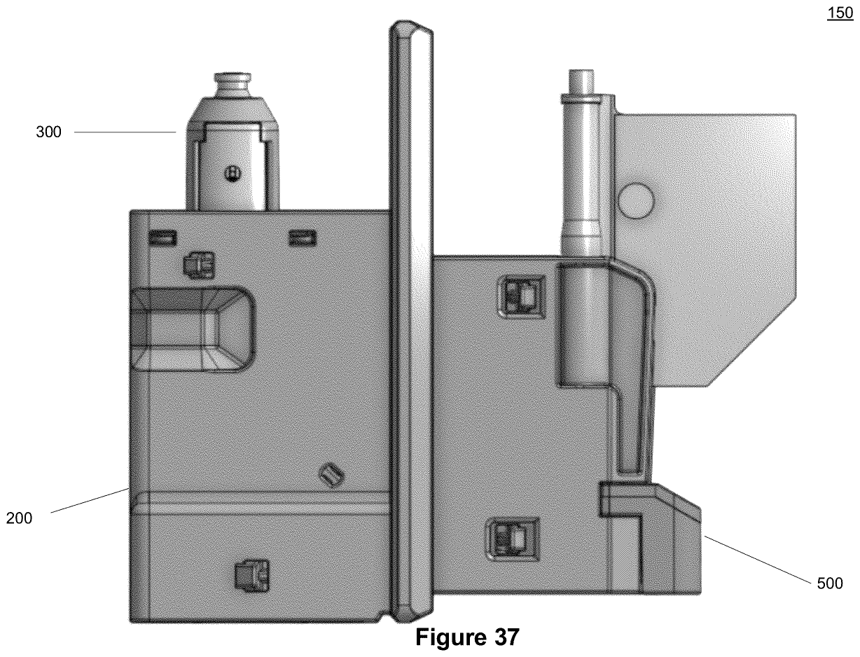

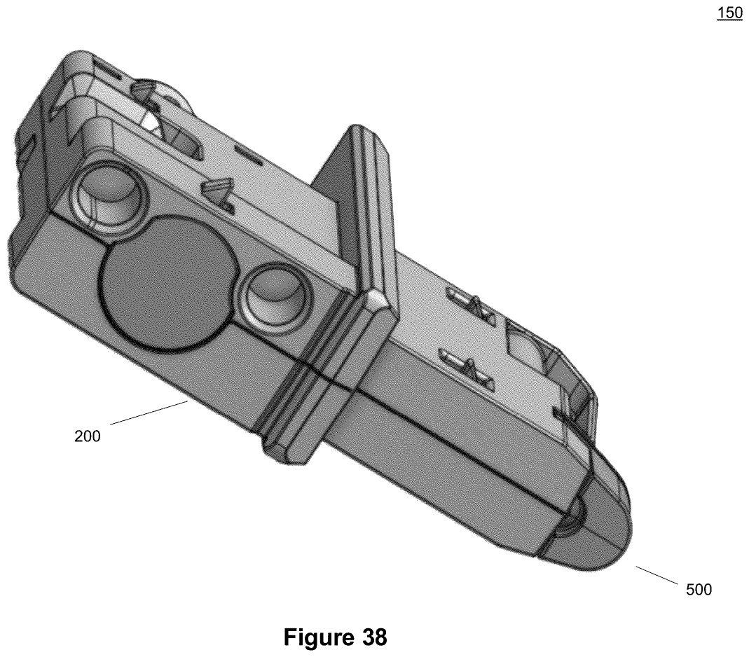

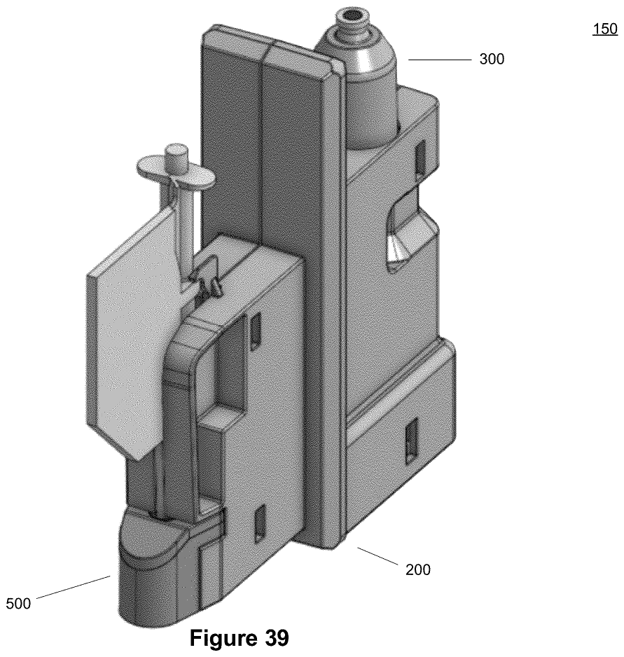

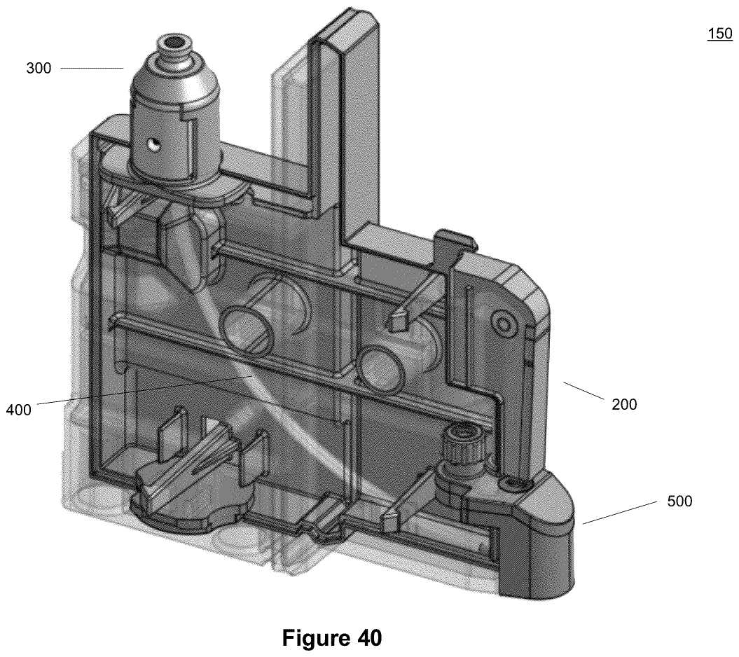

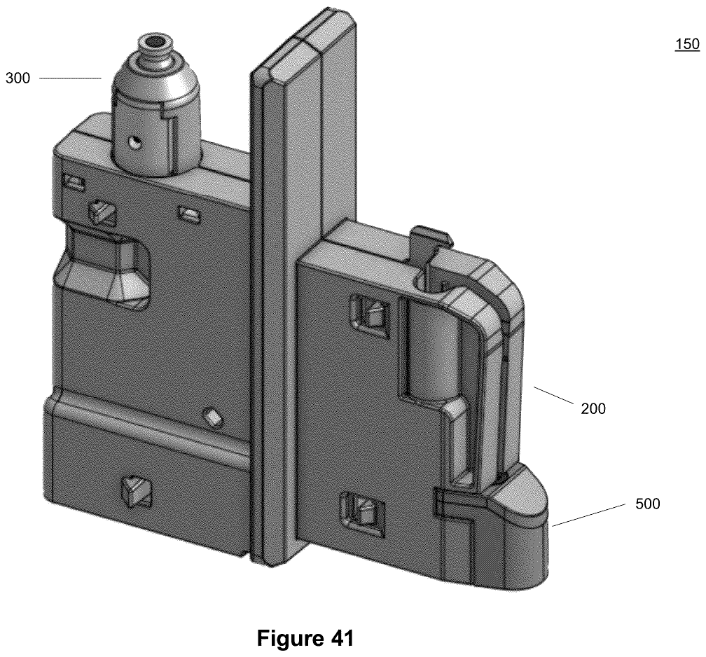

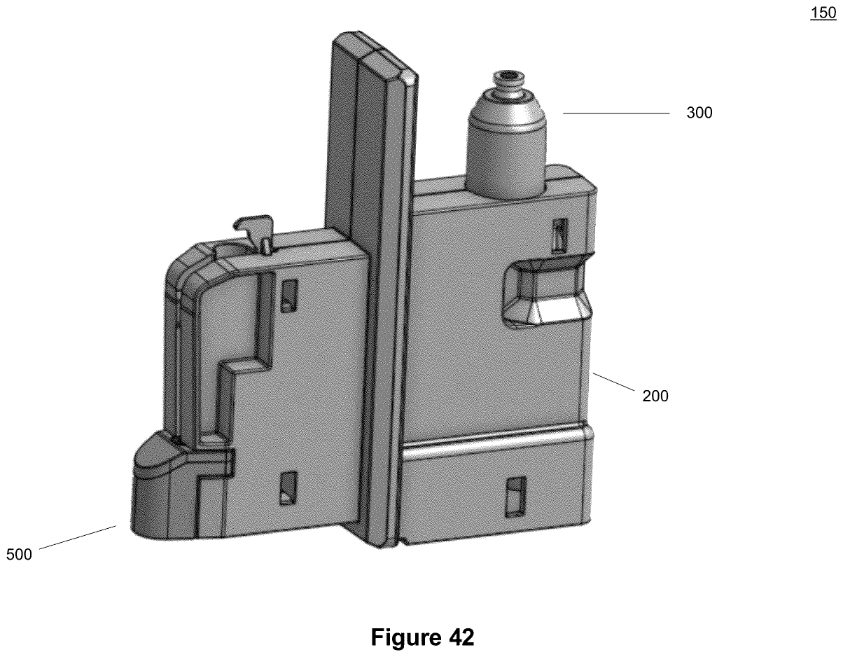

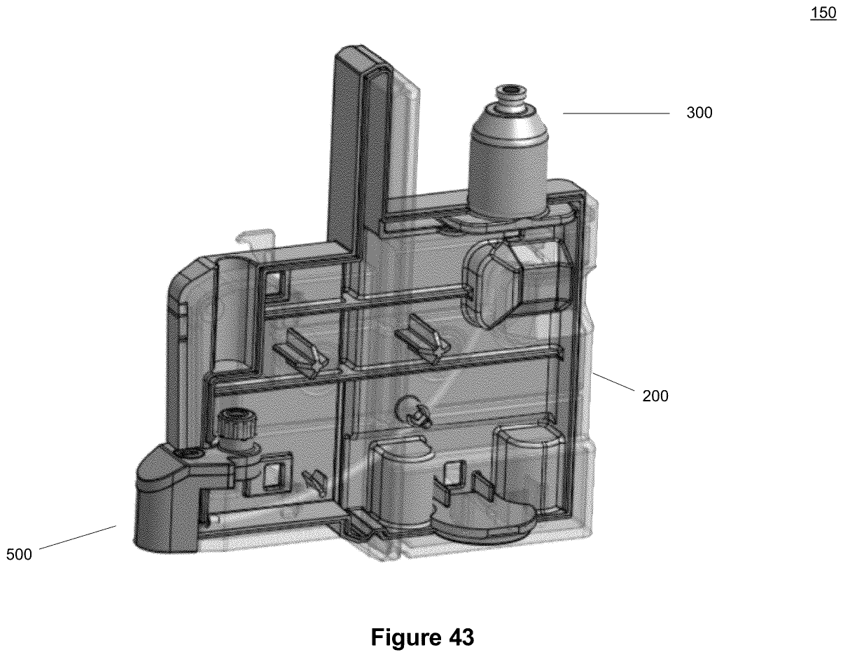

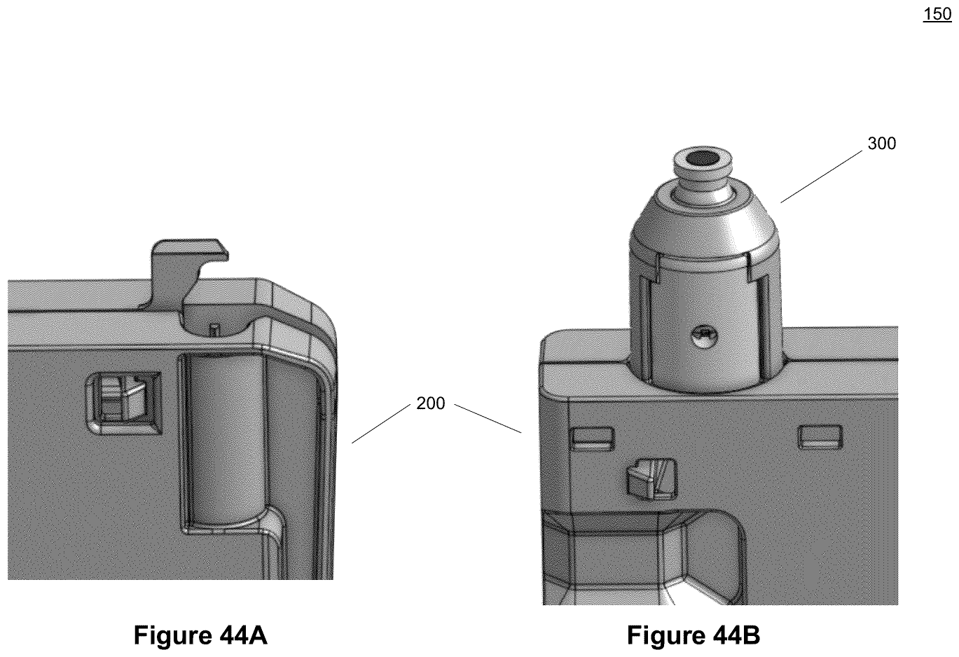

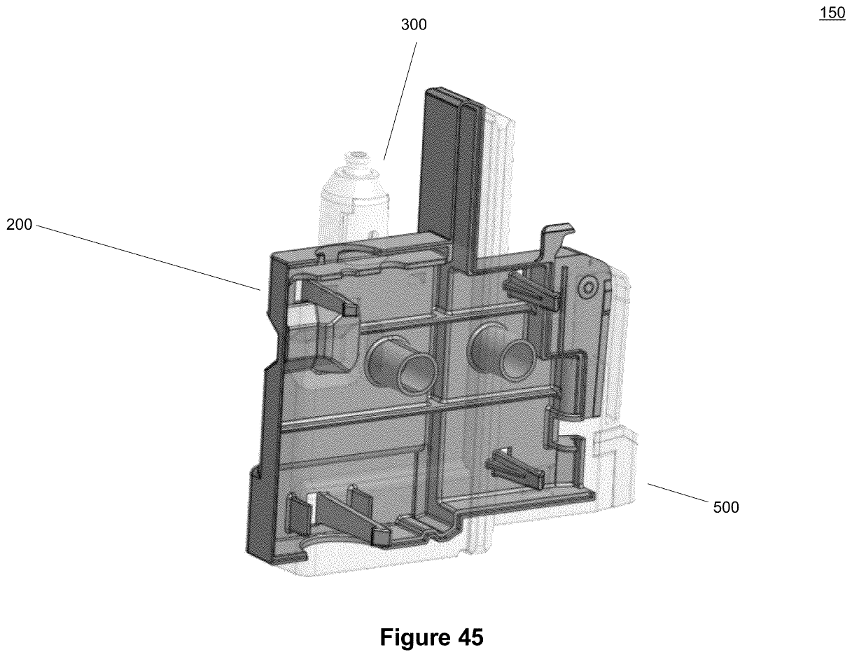

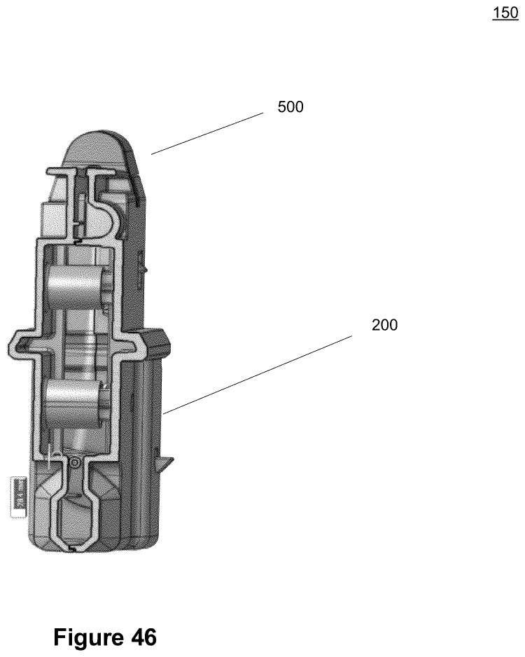

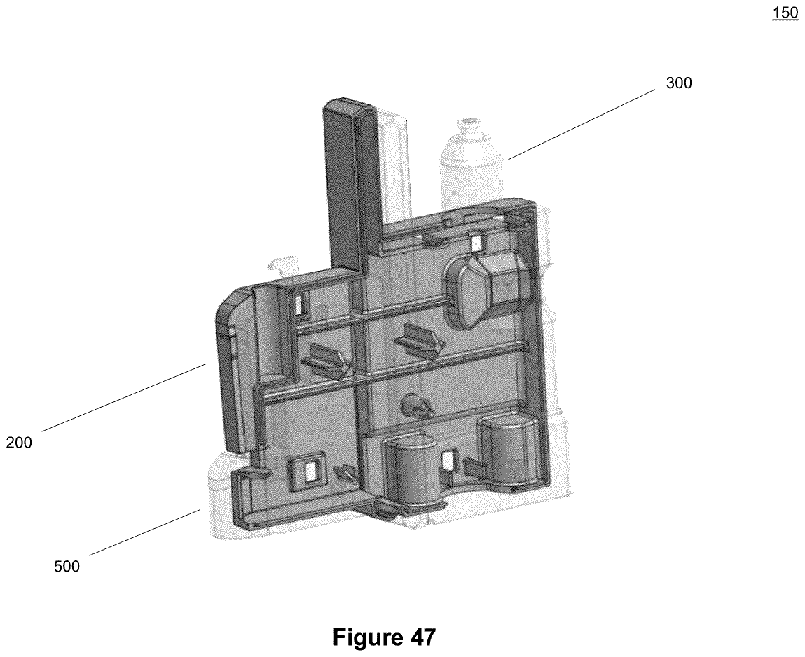

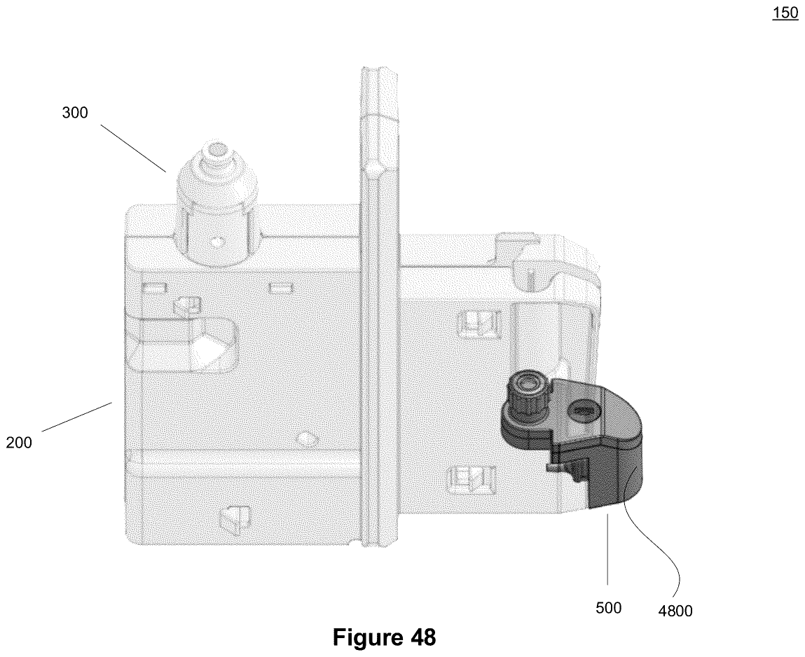

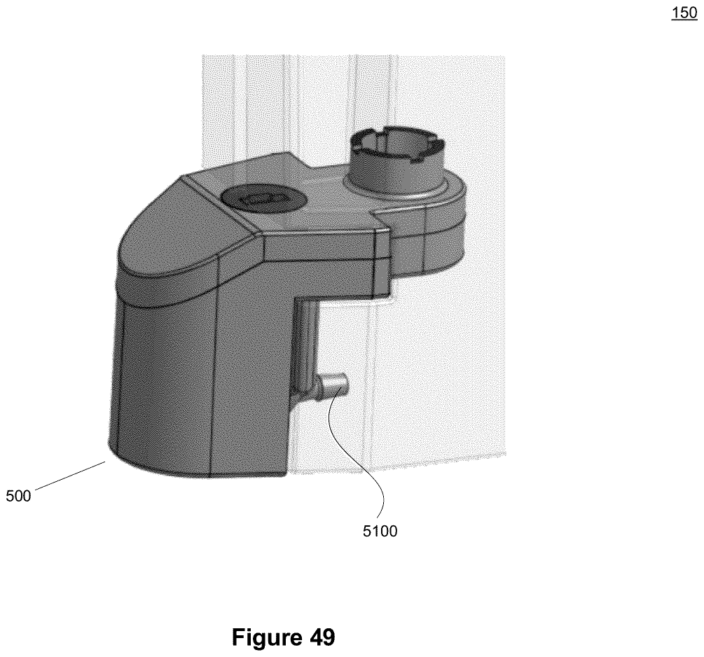

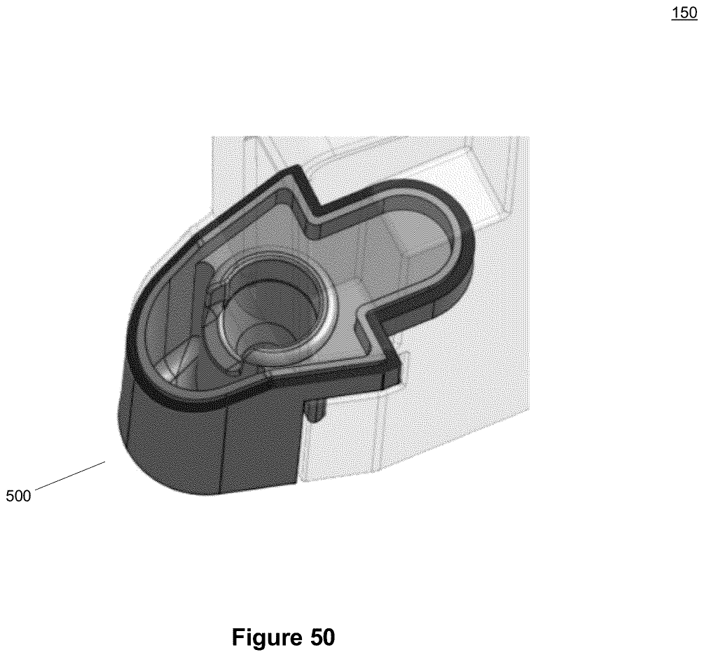

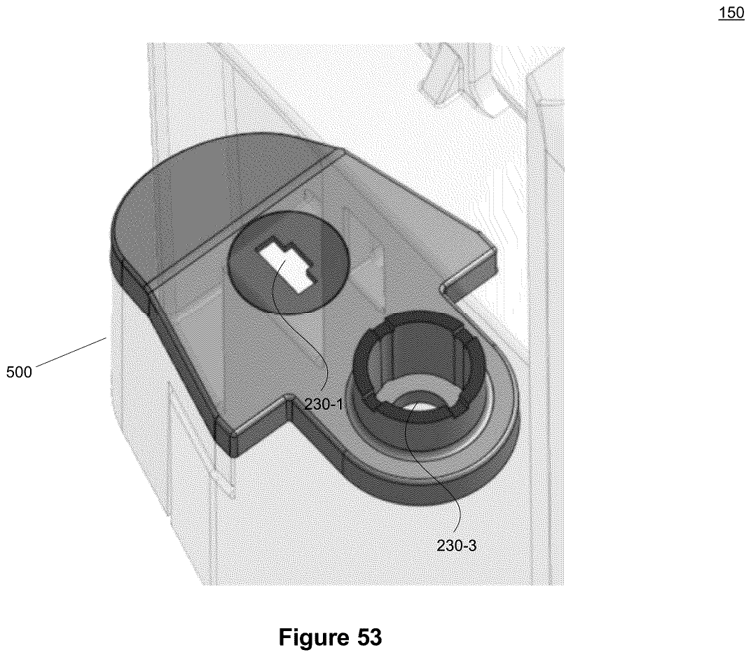

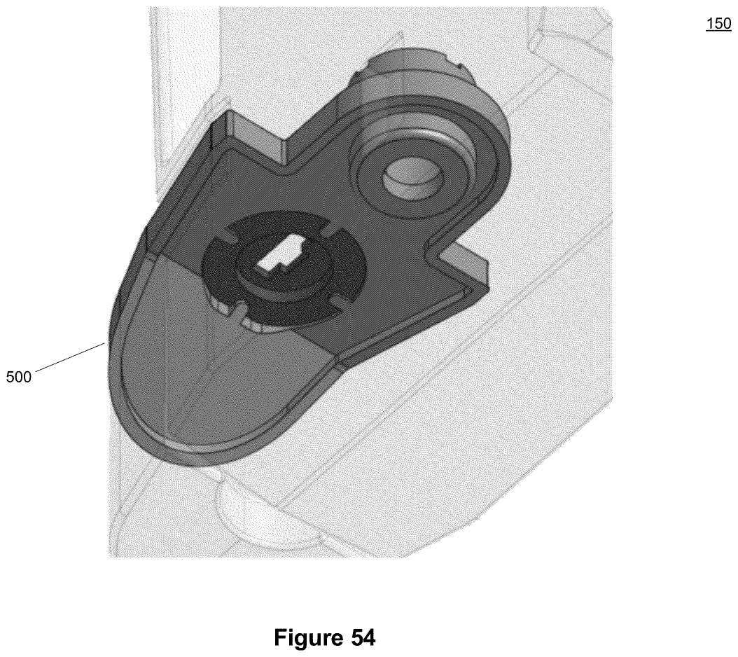

Referring to FIGS. 4-54, in some embodiments, an apparatus (e.g., apparatus 150 of FIG. 4, apparatus 150 of FIG. 5, etc.) of the present disclosure is provided. In some embodiments, the apparatus 150 is configured to accommodate and/or control a liquid, such as a first fluid flowing through some or all of the apparatus 150. For instance, in some embodiments, the apparatus 150 is configured to allow a predetermined volume of the liquid to flow from a first portion of the apparatus to a second portion of the apparatus, such as to an interior of a second device 600 in fluidic communication with the apparatus 150. However, the present disclosure is not limited thereto. In some embodiments, the liquid of the apparatus includes a biological sample, such as a plurality of viable cells. By way of non-limiting example, in some embodiments, the apparatus 150 is configured for processing a biological sample, such as by removably coupling with a sample processing device.

In some embodiments, the apparatus 150 includes a housing 200. In some embodiments, the housing 200 is configured to house and removably couple with one or more devices, such as at least two devices, at least three devices, or at least four devices different from the apparatus 150. For instance, in some embodiments, a first portion 3400-1 of the housing 200 is configured to removably couple with a first device and a second portion 3400-2 of the housing is configured to removably couple with a second device 600 different from the first device 300, providing structural rigidity to the apparatus 150. By way of non-limiting example, in some embodiments, the first device 300 includes a first reservoir that supplies a flow of the liquid to the apparatus and the second device 600 includes a second reservoir that receives some or all of the liquid supplied to the apparatus. In some embodiments, the first portion of the housing is on first side of the housing and the second portion of the housing forms a second side of the housing.

In some embodiments, the housing 200 is formed a three-dimensional monolithic structure, such a seam-less structure. However, the present disclosure is not limited thereto. In some embodiments, the housing 200 includes a first portion having one or more mating structures and a second portion having one or more corresponding mating structures, which allows the first and second portions to couple together and collectively form the housing 200. By way of non-limiting example, in some embodiments, the one or more mating structures of the housing 200 allow for toolless snap fit coupling with the one or more corresponding mating structures of the second portion of the housing, to allow for simple and fast assembly of the apparatus. As another non-limiting example, in some embodiments, the one or more mating structures comprise one or more latches and/or one or more hinges that engage with a corresponding groove or surface of the housing 200.

The housing includes a first surface 210-1 and a second surface 210-2 different from the first surface. In some embodiments, the first surface 210-1 and the second surface 210-2 are disposed on opposing sides of the housing 200. In some embodiments, the first surface 210-1 and the second surface 210-2 are disposed on a same side of the housing 200. By way of non-limiting example, in some embodiments, the first surface 210-1 and the second surface 210-2 are disposed on an upper end portion of the housing 200, which allows for the liquid to flow along a single plane from the first surface 210-1 to the second surface 210-2. However, the present disclosure is not limited thereto.

In some embodiments, the housing 200 includes a third surface (e.g., third surface 210-3 of FIG. 4). In some embodiments, the third surface includes a protrusion extending upwardly from the housing 200. For instance, in some embodiments, the protrusion of the third housing 210-3 interposes between the first and second surfaces 210 of the housing 200. In some embodiments, the protrusion comprises a surface orthogonal, or perpendicular, to the first surface and/or the second surface, which allows the third surface to block a portion of light from interfacing with the first surface or the second surface. For instance, in some embodiments, the liquid is photo-sensitive (e.g., one or more viable cells accommodated by the liquid deteriorate when exposed to light for a period of time.

In some embodiments, the housing 200 includes a fourth surface 210-4 that forms a base of the housing. In some embodiments, the base 210-4 has planar shape, an oblong shape, a circular shape, or a combination thereof. For instance, in some embodiments, the base includes a circular shape or substantially circular shape. In some embodiments, the base has other regular or irregular shapes. In some embodiments, the base is planar or substantially planar. The base is characterized by a first dimension (e.g., a width, a diameter) “D1” and a second dimension (e.g., a thickness) “D2.”

In some embodiments, the first surface 210 includes a first port 230-1, which allows access to an interior of the housing 200. In some embodiments, the first port 230-1 is disposed at a first height (e.g., height L1 of FIG. 5), such as a maximum height of the housing 200. For instance, in some embodiments, the first height L1 provides a potential energy for causing the liquid to flow through the apparatus 150 once received at the first port 230-1. In some embodiments, the first port defines an opening extending through the first portion of the housing and the second portion of the housing. In some embodiments, a diameter of the first port is greater than a diameter of the first device, which allows some or all of the first device to extend, at least in part, into the interior of the apparatus, such as a first channel 240-1. However, the present disclosure is not limited thereto.

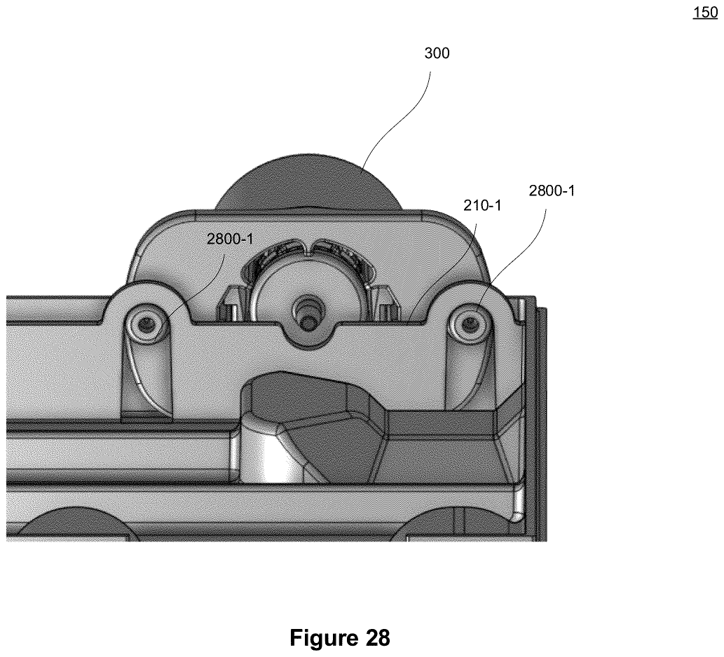

In some embodiments, the first surface 210 includes one or more grooves (e.g., one or more grooves 2800 of FIG. 28). In some embodiments, each groove 2800 in the one or more grooves is configured to receive a corresponding portion of the port body 300 and/or the second device, which allows for removably coupling the port body 300 and/or the second device to the apparatus. Moreover, in some embodiments, the groove includes a length greater than a length of the corresponding portion of the port body 300 and/or the second device, which forms a gap between an edge portion of the groove and an exterior surface of the corresponding portion of the port body 300 and/or the second device. In this way, in some such embodiments, the port body 300 and/or the second device has translational and/or rotational tolerances with respect to the first surface, allowing for slight relative movement of the port body 300 and/or the second device without disrupting the fluidic communication provided by the first channel 400-1 from the second device to the first chamber 240-1. However, the present disclosure is not limited thereto.

In some embodiments, the first port 230-1 includes a first diameter that is configured to receive an end portion of a first device 300, such as a port body 300 and/or a syringe 300. In some embodiments, the first device 300 is configured to control a flow of the liquid through the housing of the apparatus 150. By way of non-limiting example, in some embodiments, the first device 300 is configured to provide a first volume of the liquid by generating a pressure differential between an interior of the first port 230-1 and an environment. However, the present disclosure is not limited thereto.

In some embodiments, the first port 230-1 is configured to accommodate a port body 300. In some embodiments, the port body 300 includes a base and a stem extending from the base. In some embodiments, the port body 300 includes a bore extending from an upper end portion of the stem to a lower end portion of the base and configured for receiving at least a portion of a second device, such a syringe that accommodates the liquid. In some embodiments, a tip is disposed at a free end portion of the stem and configured for guiding the third device. Additional details and information regarding the port body 300 is found at United States Patent Publication No.: 2025-0224059 A, entitled “Apparatus for Facilitating Automated Connection,” filed Jan. 6, 2025, and U.S. Pat. No.: 12,479,112, entitled “Apparatus for Facilitating Automated Connection,” filed Jan. 6, 2025, each of which is hereby incorporated by reference in its entirety for all purposes.

In some embodiments, the second surface 210-2 includes a second port 230-2. In some embodiments, the second port 230-2 defines a first chamber 240-1. In some embodiments, the first chamber comprises a conical shape, a cylindrical shape, a circular shape, or a combination thereof. By way of non-limiting example, in some embodiments, a first diameter of a lower end portion of the first chamber 240-1 is less than a second diameter of an upper end portion of the first chamber 240-1. In some embodiments, an interior surface of the first chamber 240-1 includes a slope, which allows for guiding an exterior surface of a second device towards the lower end portion of the first chamber. Moreover, in some embodiments, the first chamber 240-1 has a third diameter at an intermediate portion interposing between the lower end portion and the upper end portion of the first chamber. In some embodiments, a diameter of the second device is greater than the first diameter and less than or equal to the third diameter, which allows the second device to be received by the first chamber 240-1 while preventing a lower end portion of the second device from interfacing with the lower end portion of the first chamber 240-1. Moreover, in some embodiments, a second end portion 402-2 of the first channel 400-1 is in fluidic communication with the lower end portion of the first chamber 400-1. Accordingly, in some such embodiments, by preventing the lower end portion of the second device from interfacing with the lower end portion of the first chamber 240-1, the lower end portion of the second device does not block or retard flow through the second end portion 402-2 of the first channel 400-1, such as by obstructing some or all of the flow flowing from the second end portion 402-2 of the first channel 400-1 and into the lower end portion of the second device.

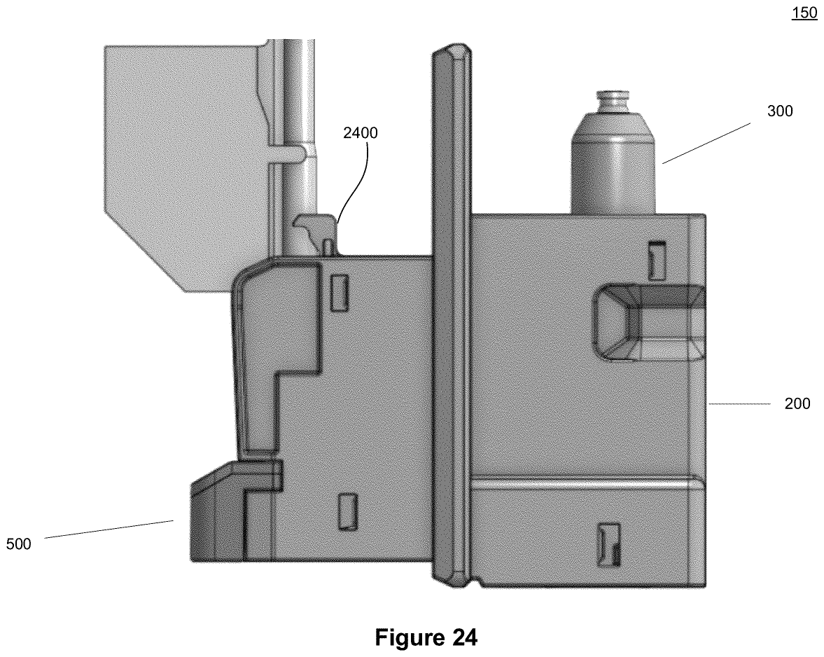

Furthermore, in some embodiments, the second port 230-3 is configured to restrict movement of the second device 600, such as restricting at least one translational degree of freedom of the second device 600 and/or restricting at least one rotational degree of freedom of the second device 600. For instance, in some embodiments, the second port 230-2 allows for receiving the second device in a first direction parallel to a central axis of the first chamber 240-1 and releasing the second device in a second direction opposite the first direction and parallel to the central axis of the first chamber 240-1. By way of non-limiting example, in some embodiments, the second port 230-2 allows for vertical displacement of the second device, such as to insert the second device 600 into the first chamber 240-1 and/or remove the second device 600 from the first chamber 240-1. However, the present disclosure is not limited thereto. In some embodiments, the second surface of the housing includes a protrusion 2400 (e.g., protrusion 2400 of FIG. 24), that is configured to interface with a corresponding surface of the second device 600, which allows the second device to be removably coupled to the apparatus 600. For instance, in some embodiments, the protrusion 2400 not only removably couples the second device 600 to the apparatus but also restricts at least one rotational degree of freedom and at least one translational degree of freedom of the second device 600. However, the present disclosure is not limited thereto. In some embodiments, the protrusion 2400 restricts the at least one rotational degree of freedom, such as a first rotational degree of free associated with a roll the second device and/or a second rotational degree of freedom associated with a pitch of the second device.

In some embodiments, the second port 230-2 is defined at a second height L2 of the housing 200. In some embodiments, the second height L2 is less than the first height L1 of the first port 230-1, which creates a potential energy difference between the first port 230-1 and the second port 230-2. In some embodiments, a difference between the first height L1 and the second height L2 allows for the liquid to flow, at least in part, from the first port 230-1 to the second port 230-2 under the force of gravity, such as without using an external pump. However, the present disclosure is not limited thereto.

In some embodiments, the second port includes a second diameter configured to receive an end portion of a second device 600. In some embodiments, the second device 600 is configured to receive a flow of the liquid provided through the housing, such as by drawing some or all of the fluid received by the first chamber 240-1 from the first channel 400-1. By way of non-limiting example, in some embodiments, the first chamber 240-1 is configured to accommodate a predetermined volume of the liquid and the second device comprises an interior channel that is configured to receive the predetermined volume of the liquid. In some such embodiments, the interior channel of the second device is in fluidic communication with the first chamber 240-1 by way of the second port 230-1. However, the present disclosure is not limited thereto. In this way, in some embodiments, the first chamber 240-1 defines a first reservoir that includes an interior volume configured to accommodate both the liquid and portion of the second device 600, such as a corresponding port of the second device 600.

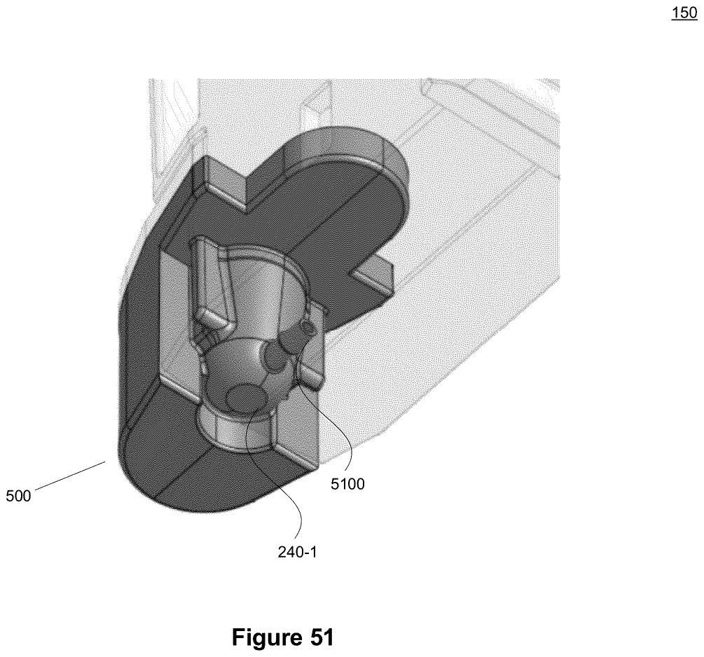

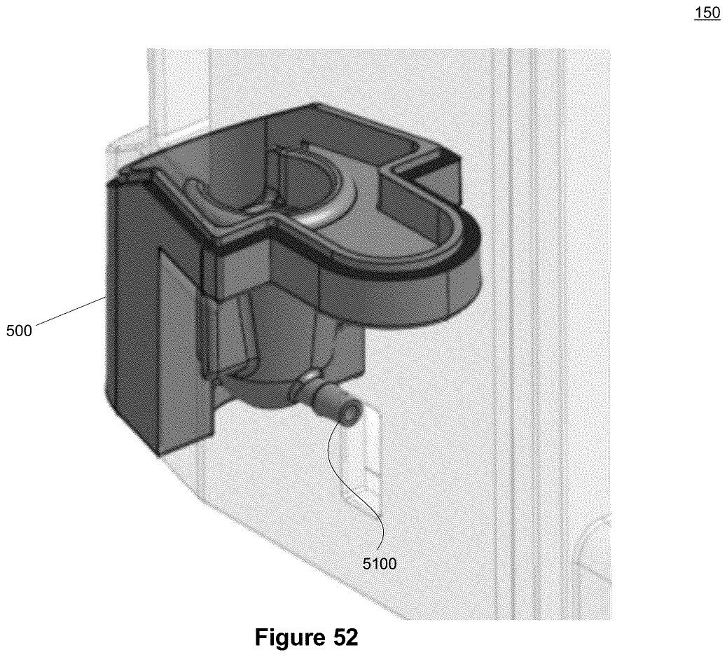

In some embodiments, a first channel (e.g., channel 400-1 of FIG. 5) is disposed in an interior of the housing 200. In some embodiments, the first channel 400-1 includes a first end portion 402-1 that is in fluidic communication with the first port 230-1, which allows the first channel 400-1 to receive and/or provide a flow of fluid. Moreover, in some embodiments, the first channel includes the second end portion 402-2 in fluidic communication with a first portion of the first chamber 240-1, such as a lower end portion of the first chamber 240-1. By way of non-limiting example, referring to FIGS. 51-52, in some embodiments, a separate housing 4800 of the first chamber 240-1 includes a protrusion 5100 comprising an opening, in which the protrusion is configured to receive the tubing to provide fluidic communication between the first port and the first chamber.

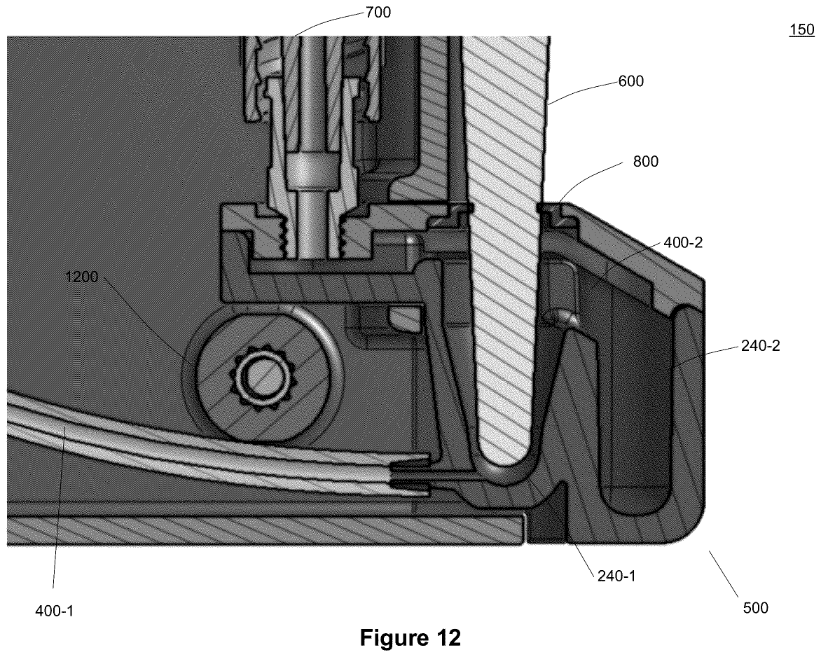

In some embodiments, the first channel is formed integrally with the housing, such as from one or more internal grooves or conduits within the housing 200. However, the present disclosure is not limited thereto. For instance, in some embodiments, the first channel 400-1 includes a tubing that fluidically couples the first chamber 240-1 and the first port 230-1 together. In some embodiments, an interior of the housing includes one or more protrusions 1200 configured to interface with an exterior surface of the tubing, such as to support the tubing, route the tubing, prevent excessive deformation of the tubing, and/or the like. For instance, referring to FIG. 12, in some embodiments, a surface of the protrusion 1200 includes a curvature that corresponds to an exterior surface of the tubing, allowing for the protrusion to interface with a length of the exterior surface of the tubing.

In some embodiments, a channel 400 of the apparatus 150 extends monotonically in a first direction relative to a gravitational vector. In some embodiments, each channel 400 of the apparatus 150 extends monotonically in a first direction relative to a gravitational vector. In some embodiments, the first channel 400-1 and the second channel 400-2 each extends monotonically in a first direction relative to a gravitational vector. In some embodiments, the first channel 400-1 extends monotonically in a first direction relative to a gravitational vector and the second channel 400-2 extends monotonically in a second direction relative to a gravitational vector, different from the first direction. In some embodiments, the first channel 400 of the apparatus defines a corresponding continuous flow path from an upstream portion of the apparatus at the first port to a downstream portion of the apparatus at the first chamber. In some embodiments, the first channel 400-1 extends downwardly and laterally through the housing from the first port to the first chamber. In some embodiments, the first channel includes a bent or curved portion, which allows for accommodating various vertical displacement(s) within the housing.

In some embodiments, a second channel 400-2 is disposed in the interior of the housing. In some embodiments, the second channel is formed integrally with the housing, such as from one or more internal grooves or conduits within the housing 200. However, the present disclosure is not limited thereto. For instance, in some embodiments, the second channel 400-2 includes a tubing that fluidically couples the first chamber 240-1 and the second chamber 240-2 together. However, the present disclosure is not limited thereto. In some embodiments, the second channel 400-2 extends downwardly and laterally through the housing or the separate housing 4800 from the second portion of the first chamber to the first portion of the second chamber.

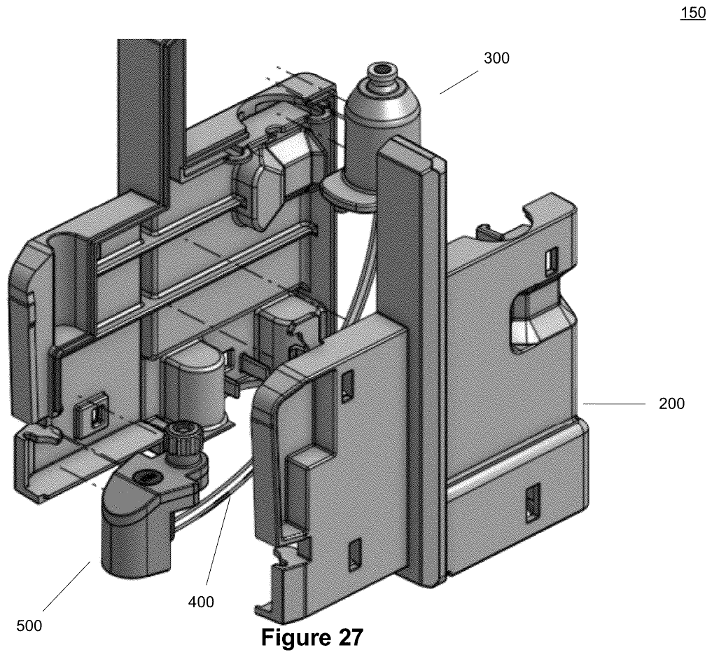

In some embodiments, the second chamber and/or the first chamber is removably coupled to the housing 200. For instance, referring to FIG. 27, in some embodiments, the first chamber and the second chamber are housed together in a separate housing 4800 from that of the apparatus, allowing reuse of the housing for various liquids that may contaminate a respective chamber. In some embodiments, the separate housing 4800 of the first and second chambers is disposed interposing between a first portion of the housing and a second portion of the housing. In some embodiments, the separate housing 4800 of the first and second chambers include a material that is optically transparent and/or translucent, allowing for visual inspection of the liquid accommodated by the first chamber and/or the second chamber. However, the present disclosure is not limited thereto.

In some embodiments, the second chamber 240-2 defines a second reservoir that includes a closed-form shape. For instance, in some embodiments, the second chamber 240-2 has a single port that provides fluidic communication between the first chamber 240-1 and the second chamber 240-2. However, the present disclosure is not limited thereto. In some embodiments, the second chamber comprises a first port that provides fluidic communication between the first chamber 240-1 via the second channel 400-2 and a second port that provides fluidic communication between the second chamber 400-2 and an external environment, such as via a filter mechanism accommodated by the second port 230-2. In some embodiments, the first channel 400 of the apparatus defines a corresponding continuous flow path from an upstream portion of the first chamber to a downstream portion of the second chamber.

In some embodiments, a first interior volume of the first chamber is greater than a second interior volume of the second chamber, which allows for an increased usable capacity of the first chamber, dampening a fluctuation caused by transient inflow or outflow events or turbulence, and/or the like.

In some embodiments, a volume of liquid accommodated by the first interior volume of the first chamber is at least between 150 and 250 μL, 150 and 200 μL, 153 and 247 μL, 153 and 197 μL, 156 and 244 μL, 156 and 194 μL, 160 and 240 μL, 160 and 190 μL, 163 and 237 μL, 163 and 187 μL, 166 and 234 μL, 166 and 184 μL, 169 and 231 μL, 169 and 181 μL, 173 and 227 μL, 173 and 177 μL, 176 and 224 μL, 179 and 221 μL, 182 and 218 μL, 185 and 215 μL, 189 and 211 μL, 192 and 208 μL, 195 and 205 μL, 198 and 202 μL, 200 and 250 μL, 203 and 247 μL, 206 and 244 μL, 210 and 240 μL, 213 and 237 μL, 216 and 234 μL, 219 and 231 μL, or 223 and 227 μL. In some embodiments, a volume of liquid accommodated by the first interior volume of the first chamber is at least 150 μL, at least 153 μL, at least 156 μL, at least 160 μL, at least 163 μL, at least 166 μL, at least 169 μL, at least 173 μL, at least 176 μL, at least 177 μL, at least 179 μL, at least 181 μL, at least 182 μL, at least 184 μL, at least 185 μL, at least 187 μL, at least 189 μL, at least 190 μL, at least 192 μL, at least 194 μL, at least 195 μL, at least 197 μL, at least 198 μL, at least 200 μL, at least 202 μL, at least 203 μL, at least 205 μL, at least 206 μL, at least 208 μL, at least 210 μL, at least 211 μL, at least 213 μL, at least 215 μL, at least 216 μL, at least 218 μL, at least 219 μL, at least 221 μL, at least 223 μL, at least 224 μL, at least 227 μL, at least 231 μL, at least 234 μL, at least 237 μL, at least 240 μL, at least 244 μL, at least 247 μL, or at least 250 μL. In some embodiments, a volume of liquid accommodated by the first interior volume of the first chamber is at most 150 μL, at most 153 μL, at most 156 μL, at most 160 μL, at most 163 μL, at most 166 μL, at most 169 μL, at most 173 μL, at most 176 μL, at most 177 μL, at most 179 μL, at most 181 μL, at most 182 μL, at most 184 μL, at most 185 μL, at most 187 μL, at most 189 μL, at most 190 μL, at most 192 μL, at most 194 μL, at most 195 μL, at most 197 μL, at most 198 μL, at most 200 μL, at most 202 μL, at most 203 μL, at most 205 μL, at most 206 μL, at most 208 μL, at most 210 μL, at most 211 μL, at most 213 μL, at most 215 μL, at most 216 μL, at most 218 μL, at most 219 μL, at most 221 μL, at most 223 μL, at most 224 μL, at most 227 μL, at most 231 μL, at most 234 μL, at most 237 μL, at most 240 μL, at most 244 μL, at most 247 μL, at most 250 μL.

In some embodiments, a volume of liquid accommodated by a first interior volume of the second chamber is at least between 150 and 250 μL, 150 and 200 μL, 153 and 247 μL, 153 and 197 μL, 156 and 244 μL, 156 and 194 μL, 160 and 240 μL, 160 and 190 μL, 163 and 237 μL, 163 and 187 μL, 166 and 234 μL, 166 and 184 μL, 169 and 231 μL, 169 and 181 μL, 173 and 227 μL, 173 and 177 μL, 176 and 224 μL, 179 and 221 μL, 182 and 218 μL, 185 and 215 μL, 189 and 211 μL, 192 and 208 μL, 195 and 205 μL, 198 and 202 μL, 200 and 250 μL, 203 and 247 μL, 206 and 244 μL, 210 and 240 μL, 213 and 237 μL, 216 and 234 μL, 219 and 231 μL, or 223 and 227 μL. In some embodiments, a volume of liquid accommodated by the first interior volume of the second chamber is at least 150 μL, at least 153 μL, at least 156 μL, at least 160 μL, at least 163 μL, at least 166 μL, at least 169 μL, at least 173 μL, at least 176 μL, at least 177 μL, at least 179 μL, at least 181 μL, at least 182 μL, at least 184 μL, at least 185 μL, at least 187 μL, at least 189 μL, at least 190 μL, at least 192 μL, at least 194 μL, at least 195 μL, at least 197 μL, at least 198 μL, at least 200 μL, at least 202 μL, at least 203 μL, at least 205 μL, at least 206 μL, at least 208 μL, at least 210 μL, at least 211 μL, at least 213 μL, at least 215 μL, at least 216 μL, at least 218 μL, at least 219 μL, at least 221 μL, at least 223 μL, at least 224 μL, at least 227 μL, at least 231 μL, at least 234 μL, at least 237 μL, at least 240 μL, at least 244 μL, at least 247 μL, or at least 250 μL. In some embodiments, a volume of liquid accommodated by the first interior volume of the second chamber is at most 150 μL, at most 153 μL, at most 156 μL, at most 160 μL, at most 163 μL, at most 166 μL, at most 169 μL, at most 173 μL, at most 176 μL, at most 177 μL, at most 179 μL, at most 181 μL, at most 182 μL, at most 184 μL, at most 185 μL, at most 187 μL, at most 189 μL, at most 190 μL, at most 192 μL, at most 194 μL, at most 195 μL, at most 197 μL, at most 198 μL, at most 200 μL, at most 202 μL, at most 203 μL, at most 205 μL, at most 206 μL, at most 208 μL, at most 210 μL, at most 211 μL, at most 213 μL, at most 215 μL, at most 216 μL, at most 218 μL, at most 219 μL, at most 221 μL, at most 223 μL, at most 224 μL, at most 227 μL, at most 231 μL, at most 234 μL, at most 237 μL, at most 240 μL, at most 244 μL, at most 247 μL, at most 250 μL.

In some embodiments, the second channel 400-2 includes a first end portion 402-1 in fluidic communication with a second portion of the first chamber 240-1, such as an upper end portion of the first chamber 240-1. Moreover, in some embodiments, the second channel 400-2 includes a second end portion 402-2 that is in fluidic communication with a first portion of a second chamber 240-2, such as an upper end portion of the second chamber 240-1.

In some embodiments, an elevation, or height, of the second portion of the first chamber 240-1 is greater than an elevation, or height, of the first portion of a second chamber 240-1, which creates a potential energy difference between the first chamber 240-1 and the second chamber 240-2. For instance, in some embodiments, the first end portion of the second channel 400-2 includes a first height and the second end portion of the second channel 400-2 includes a second height less than the first height, which creates a downward slope for the fluid to flow under gravity from the first chamber 240-1 to the second chamber 240-2. However, the present disclosure is not limited thereto. In this way, in some embodiments, the liquid is allowed to flow from the first chamber 240-1 to the second chamber 240-2 once the volume of liquid in the first chamber 240-1 exceed the elevation or height of the second portion of the first chamber 240-1. Said otherwise, in some embodiments, the second chamber 240-2 is configured to receive an overflow of the liquid from the first chamber 240-1 once a threshold volume of fluid is satisfied at the first chamber 240-1 while maintaining a predetermined volume of the liquid within the first chamber 240-1. However, the present disclosure is not limited thereto.

In some embodiments, the second port 230-2 is configured to accommodate a gasket 800 that seals an interior of the housing, at least in part, from an external environment. For instance, in some embodiments, the gasket 800 is configured to seal the first chamber 240-1 from an external environment, seal the second chamber 240-2 from the external environment, or seal both the first and second chambers from an external environment. However, the present disclosure is not limited thereto. In some embodiments, the gasket 800 includes a first surface configured to interface with an exterior surface of the second port 230-2 and a second surface configured to interface with an exterior surface of a second device 600, creating a seal between both the gasket and the second port 230-2 and the gasket and the second device 600.

In some embodiments, the first channel extends downwardly relative to a gravitational vector when the apparatus is (i) horizontal with the ground and/or (ii) rotated about a first axis of the housing by at least 45 degrees. By way of non-limiting example, in some embodiments, the apparatus 150 is operable for being spun around a rotational axis (e.g., rotated about a first axis, such as a longitudinal axis of the apparatus 150). In some embodiments, rotating the apparatus from a first orientation (e.g., a first orientation in which the first surface of the housing is co-planar with the ground) to a second orientation (e.g., a second orientation in which the first surface of the housing is parallel or substantially parallel with the ground). However, the present disclosure is not limited thereto.

In some embodiments, the apparatus 150 further includes a third channel 400-3. In some embodiments, the third channel 400-3 is disposed in the interior of the housing 200. In some embodiments, the third channel includes a first end portion in fluidic communication with the second portion of the first chamber and a second end portion in fluidic communication with a third opening of the housing.

In some embodiments, the third opening is formed in an interior of the housing.

In some embodiments, the third opening is configured to accommodate a filter mechanism 700. For instance, in some embodiments, the filter mechanism 700 is configured to allow an ingress and/or egress of fluid from the first chamber and/or the second chamber, such as to allow for air flow to enter and/or exit the first chamber and/or the second chamber to prevent a pressure surge and/or vacuum.

In some embodiments, the apparatus 150 includes a reservoir (e.g., first chamber 240-1) that is configured to accommodate a volume of the liquid and provide (e.g., supply) some or all of the liquid from the reservoir to a portion of the apparatus (e.g., second device 600). In some embodiments, the apparatus includes an interior volume for accommodating the liquid received from the reservoir. However, the present disclosure is not limited thereto. Moreover, in some embodiments, the apparatus includes a body configured to receive the liquid from the interior volume. In this way, the apparatus allows for receiving the liquid from the reservoir, accommodating the liquid within the interior of the apparatus, and providing the liquid to the body of the apparatus, such that the liquid is conveyed from the reservoir to the body within risk of contamination from an environment. However, the present disclosure is not limited thereto.

In some embodiments, the apparatus includes a device configured to evaluate one or more cells of a cellular therapy. For instance, in some embodiments, the device is configured to evaluate cells by receiving an exterior surface of the apparatus and determining a number of cells visible through a transparent portion of the second device, such as by utilizing an image cytometry process. In some embodiments, the second device is configured to accommodate 60 microliters of liquid. In some embodiments, the second device is configured to evaluate 1.4 microliters of liquid.

In some embodiments, the apparatus includes one or more docks (e.g., port bodies) configured to receive a syringe device (e.g., first device 300), which allows the one or more docks to provide a reservoir of syringes. However, the present disclosure is not limited thereto.

In some embodiments, the apparatus includes a nucleocounter cassette (e.g., second device 600).

In some embodiments, the apparatus includes an Eppendorf chamber (“EPP”) (e.g., first chamber).

In some embodiments, the apparatus is configured to maintain a portion of the second device as a closed system, such that the portion of the second device is sealed or separated from an environment. For instance, in some embodiments, the apparatus includes a seal, a gasket, an O-ring, a fluid-tight device, an adhesive (e.g., a layer of adhesive material) or a combination thereof that is disposed about an edge portion of a channel (e.g., port), which allows for preventing contamination of the liquid accommodated by the fluid reservoir.

In some embodiments, the liquid accommodated by the fluid reservoir and/or the apparatus is light sensitive and/or moisture sensitive. For instance, in some embodiments, the fluid is light sensitive with threshold time tolerance of about 1 hour and a threshold moisture tolerance of about 2 weeks (e.g., about 336 hours). Accordingly, in some embodiments, the apparatus is configured to provide light and/or moisture protection for the liquid. For instance, in some embodiments, the apparatus includes a housing having a first surface configured to shield like during storage. In some embodiments, the first surface extends vertically from a base of the apparatus.

In some embodiments, the apparatus includes one or more ports configured to receive a device, such as a syringe, a reservoir, or another apparatus of the present disclosure. For instance, in some embodiments, the port includes an opening comprising an upper end portion configured to engage with the device and a lower end portion in fluid communication with a channel (e.g., fluid channel) that is configured to convey the liquid accommodated by the device to an interior volume of the apparatus.

In some embodiments, the apparatus is configured to prevent some or all of the VCC device from interfering with a different portion of the apparatus, such as insertion into the NC-200 device. However, the present disclosure is not limited thereto.

In some embodiments, the apparatus is configured to prevent sampling fluid from reservoir in accordance with a determination the apparatus is in a first state (e.g., a powered stated), such that the liquid is allowed to flow to a separate overflow region or interior volume.

In some embodiments, a volume of liquid accommodated by an interior of the apparatus, such as an EPP chamber of the interior of the apparatus, is at least 200 microliters (200 μL) in accordance with a determination an end portion (e.g., a tip) of the channel (e.g., pipette) engages against a lower end surface of the reservoir. In some embodiments, a volume of liquid accommodated by a reservoir is at least between 150 and 250 μL, 150 and 200 μL, 153 and 247 μL, 153 and 197 μL, 156 and 244 μL, 156 and 194 μL, 160 and 240 μL, 160 and 190 μL, 163 and 237 μL, 163 and 187 μL, 166 and 234 μL, 166 and 184 μL, 169 and 231 μL, 169 and 181 μL, 173 and 227 μL, 173 and 177 μL, 176 and 224 μL, 179 and 221 μL, 182 and 218 μL, 185 and 215 μL, 189 and 211 μL, 192 and 208 μL, 195 and 205 μL, 198 and 202 μL, 200 and 250 μL, 203 and 247 μL, 206 and 244 μL, 210 and 240 μL, 213 and 237 μL, 216 and 234 μL, 219 and 231 μL, or 223 and 227 μL. In some embodiments, a volume of liquid accommodated by a reservoir is at least 150 μL, at least 153 μL, at least 156 μL, at least 160 μL, at least 163 μL, at least 166 μL, at least 169 μL, at least 173 μL, at least 176 μL, at least 177 μL, at least 179 μL, at least 181 μL, at least 182 μL, at least 184 μL, at least 185 μL, at least 187 μL, at least 189 μL, at least 190 μL, at least 192 μL, at least 194 μL, at least 195 μL, at least 197 μL, at least 198 μL, at least 200 μL, at least 202 μL, at least 203 μL, at least 205 μL, at least 206 μL, at least 208 μL, at least 210 μL, at least 211 μL, at least 213 μL, at least 215 μL, at least 216 μL, at least 218 μL, at least 219 μL, at least 221 μL, at least 223 μL, at least 224 μL, at least 227 μL, at least 231 μL, at least 234 μL, at least 237 μL, at least 240 μL, at least 244 μL, at least 247 μL, or at least 250 μL. In some embodiments, a volume of liquid accommodated by a reservoir is at most 150 μL, at most 153 μL, at most 156 μL, at most 160 μL, at most 163 μL, at most 166 μL, at most 169 μL, at most 173 μL, at most 176 μL, at most 177 μL, at most 179 μL, at most 181 μL, at most 182 μL, at most 184 μL, at most 185 μL, at most 187 μL, at most 189 μL, at most 190 μL, at most 192 μL, at most 194 μL, at most 195 μL, at most 197 μL, at most 198 μL, at most 200 μL, at most 202 μL, at most 203 μL, at most 205 μL, at most 206 μL, at most 208 μL, at most 210 μL, at most 211 μL, at most 213 μL, at most 215 μL, at most 216 μL, at most 218 μL, at most 219 μL, at most 221 μL, at most 223 μL, at most 224 μL, at most 227 μL, at most 231 μL, at most 234 μL, at most 237 μL, at most 240 μL, at most 244 μL, at most 247 μL, at most 250 μL.

In some embodiments, one or more portions of the apparatus include a biocompatible plastic material, such as a USP Class VI material and/or the like.

Referring briefly to FIG. 15-24, in some embodiments, the present disclosure provides a method for controlling liquid, such as for controlling a liquid received by an apparatus and/or provided by the apparatus.

In some embodiments, the articulated handling robot places the apparatus into an aspiration station.

In some embodiments, the articulated handling robot places a syringe device 300 into the apparatus and injects some or all of the liquid accommodated by the syringe device into the interior volume of the apparatus.

In some embodiments, the articulated handling robot depresses plunger of o hard stop to draw the liquid into an interior channel of the second device.

In some embodiments, the articulated handling robot traverses the apparatus and tilts the apparatus forward, towards the second chamber of the apparatus to allow excess liquid to enter the second chamber via the second channel from the first chamber.

In some embodiments, the articulated handling robot pulls the plunger of the syringe device to remove the liquid from first chamber after aspiration.

In some embodiments, the articulated handling robot couples the apparatus to a third device. In some embodiments, an orifice of the apparatus prevents liquid uptake.

In some embodiments, the liquid is drawn into an optical window of the apparatus. In some embodiments, the third device evaluates a cell count accommodated by a portion of the liquid.

### References Cited and Alternative Embodiments

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. An apparatus for controlling a liquid, the apparatus comprising
a housing comprising a first surface and a second surface different from the first surface, wherein
the first surface comprises a first port disposed at a first height,
the second surface comprises a second port defining a first chamber, the second port defined at a second height, less than the first height,
a first channel disposed in an interior of the housing, the first channel comprising a first end portion in fluidic communication with the first port and a second end portion in fluidic communication with a first portion of the first chamber, and
a second channel disposed in the interior of the housing, the second channel comprising a first end portion in fluidic communication with a second portion of the first chamber and a second end portion in fluidic communication with a first portion of a second chamber.

2. The apparatus of claim 1, wherein the first port comprises a first diameter configured to receive an end portion of a first device, and wherein the first device is configured to control a flow of the liquid through the housing.

3. The apparatus of claim 1, wherein the second port comprises a second diameter configured to receive an end portion of a second device, and wherein the second device is configured to receive a flow of the liquid through the housing.

4. The apparatus of claim 1, wherein the second port is configured to accommodate a gasket comprising a first surface configured to interface with an exterior surface of the second port and a second surface configured to interface with an exterior surface of a second device.

5. The apparatus of claim 1, wherein the first chamber defines a first reservoir comprising an interior volume configured to accommodate both the liquid and a second device.

6. The apparatus of claim 1, wherein the second chamber defines a second reservoir comprising a closed-form shape.

7. The apparatus of claim 1, wherein the first end portion of the second channel comprises a first height and the second end portion of the second channel comprises a second height less than the first height.

8. The apparatus of claim 1, wherein the first channel defines a continuous fluid flow path between an upstream portion at the first port and a down stream portion at the first portion of the first chamber.

9. The apparatus of claim 8, wherein the first channel extends monotonically in a first direction relative to a gravitational vector.

10. The apparatus of claim 1, wherein the first channel extends downwardly relative to a gravitational vector when the apparatus is (i) horizontal with the ground and/or (ii) rotated about a first axis of the housing by at least 45 degrees.

11. The apparatus of claim 1, wherein the second channel defines a continuous fluid flow path between an upstream portion at the second portion of the first chamber and a down stream portion at the first portion of the second chamber.

12. The apparatus of claim 10, wherein the second channel extends monotonically in a first direction relative to a gravitational vector.

13. The apparatus of claim 1, wherein a first interior volume of the first chamber is greater than a second interior volume of the second chamber.

14. The apparatus of claim 1, wherein the first portion of the second chamber comprises the sole fluid opening of the second chamber.

15. The apparatus of claim 1, wherein the apparatus further comprises a third channel disposed in the interior of the housing, the second channel comprising a first end portion in fluidic communication with the second portion of the first chamber and a second end portion in fluidic communication with a third opening of the housing.

16. The apparatus of claim 15, wherein the third opening is formed in an interior of the housing.

17. The apparatus of claim 15, wherein the third opening is configured to accommodate a filter mechanism.

18. The apparatus of claim 1, the first channel comprises a tubing, and wherein the second channel is formed by at least a first interior surface of the housing.

19. The apparatus of claim 1, wherein the housing is configured to mate to a corresponding external surface of a third device.

20. The apparatus of claim 1, wherein the first opening comprises
a port body comprising:
a base;
a stem extending from the base;
a bore extending from an upper end portion of the stem to a lower end portion of the base and configured for receiving at least a portion of the first device;
a tip disposed at a free end portion of the stem and configured for guiding the second device when connecting the second device and the first device; and
one or more first anti-rotation members disposed at the base; and
a retainer comprising:
a first retaining member having a first surface;
a second retaining member coupled or formed with the first retaining member and having a second surface spaced apart from the first surface of the first retaining member in an axial direction of the port body, wherein the base of the port body is disposed between the first surface of the first retaining member and the second surface of the second retaining member;
a first circular or substantially circular through-hole disposed on the first retaining member and having a diameter larger or substantially larger than an outer diameter of the stem to allow the stem of the port body to pass through and to move relative to the first retaining member; and
one or more second anti-rotation members disposed at the first retaining member or the second retaining member and coupled with the one or more first
anti-rotation members to restrict the port body from rotating relative to the retainer,
wherein the port body is movable translationally relative to the retainer in a plane substantially perpendicular to the axial direction of the port body.
